SEQUENCE LISTING (66 sequences)

SEQ ID NO: 1 - DNA, 35935 bp, Adenovirus type 5
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgtgtagtgtatttatacccggtgagttcctcaagaggccactcttgagtgccagcgagtagagttttctcctccgagccgctccgacaccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaatcggggttggggttattctctgtcttgtgattctctttattcttatactaacgcttctctgcctaaggctcgccgcctgctgtgtgcacatttgcatttattgtcagctttttaaacgctggggtcgccacccaagatgattaggtacataatcctaggtttactcacccttgcgtcagcccacggtaccacccaaaaggtggattttaaggagccagcctgtaatgttacattcgcagctgaagctaatgagtgcaccactcttataaaatgcaccacagaacatgaaaagctgcttattcgccacaaaaacaaaattggcaagtatgctgtttatgctatttggcagccaggtgacactacagagtataatgttacagttttccagggtaaaagtcataaaacttttatgtatacttttccattttatgaaatgtgcgacattaccatgtacatgagcaaacagtataagttgtggcccccacaaaattgtgtggaaaacactggcactttctgctgcactgctatgctaattacagtgctcgctttggtctgtaccctactctatattaaatacaaaagcagacgcagctttattgaggaaaagaaaatgccttaatttactaagttacaaagctaatgtcaccactaactgctttactcgctgcttgcaaaacaaattcaaaaagttagcattataattagaataggatttaaaccccccggtcatttcctgctcaataccattcccctgaacaattgactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgattaaatgagacatgattcctcgagtttttatattactgacccttgttgcgcttttttgtgcgtgctccacattggctgcggtttctcacatcgaagtagactgcattccagccttcacagtctatttgctttacggatttgtcaccctcacgctcatctgcagcctcatcactgtggtcatcgcctttatccagtgcattgactgggtctgtgtgcgctttgcatatctcagacaccatccccagtacagggacaggactatagctgagcttcttagaattctttaattatgaaatttactgtgacttttctgctgattatttgcaccctatctgcgttttgttccccgacctccaagcctcaaagacatatatcatgcagattcactcgtatatggaatattccaagttgctacaatgaaaaaagcgatctttccgaagcctggttatatgcaatcatctctgttatggtgttctgcagtaccatcttagccctagctatatatccctaccttgacattggctggaaacgaatagatgccatgaaccacccaactttccccgcgcccgctatgcttccactgcaacaagttgttgccggcggctttgtcccagccaatcagcctcgccccacttctcccacccccactgaaatcagctactttaatctaacaggaggagatgactgacaccctagatctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccttgacaaaagaacccacactgattatgacacgcatactcggagctatgctaaccagcgtagccccgatgtaagctttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttcacagcggcagcctaacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg

SEQ ID NO: 2 - DNA, 21 bp, Artificial Sequence
  Description of Artificial Sequence Primer
gggtggaaagccagcctcgtg

SEQ ID NO: 3 - DNA, 21 bp, Artificial Sequence
  Description of Artificial Sequence Primer
acccgcaggcgtagagacaac

SEQ ID NO: 4 - DNA, 41 bp, Artificial Sequence
  Description of Artificial Sequence Primer
agatcaaagggattaagatcaaagggccaccacctcattat

SEQ ID NO: 5 - DNA, 48 bp, Artificial Sequence
  Description of Artificial Sequence Primer
tccctttgatctccaaccctttgatctagtcctatttatacccggtga

SEQ ID NO: 6 - DNA, 44 bp, Artificial Sequence
  Description of Artificial Sequence Primer
tccctttgatctccactagtgtgaattgtagttttcttaaaatg

SEQ ID NO: 7 - DNA, 27 bp, Artificial Sequence
  Description of Artificial Sequence Primer
gaactagtagtaaatttgggcgtaacc

SEQ ID NO: 8 - DNA, 25 bp, Artificial Sequence
  Description of Artificial Sequence Primer
acgctagcaaaacacctgggcgagt

SEQ ID NO: 9 - DNA, 20 bp, Artificial Sequence
  Description of Artificial Sequence Primer
cattttcagtcccggtgtcg

SEQ ID NO: 10 - DNA, 20 bp, Artificial Sequence
  Description of Artificial Sequence Primer
accgaagaaatggccgccag

SEQ ID NO: 11 - DNA, 25 bp, Artificial Sequence
  Description of Artificial Sequence Primer
tctgtaatgttggcggtgcaggaag

SEQ ID NO: 12 - DNA, 20 bp, Artificial Sequence
  Description of Artificial Sequence Primer
atggctaggaggtggaagat

SEQ ID NO: 13 - DNA, 20 bp, Artificial Sequence
  Description of Artificial Sequence Primer
gtgtcggagcggctcggagg

SEQ ID NO: 14 - DNA, 21 bp, Artificial Sequence
  Description of Artificial Sequence Primer
caggtcctcatatagcaaagc

SEQ ID NO: 15 - DNA, 20 bp, Artificial Sequence
  Description of Artificial Sequence Primer
tgtctgaacctgagcctgag

SEQ ID NO: 16 - DNA, 18 bp, Artificial Sequence
  Description of Artificial Sequence Primer
catctctacagcccatac

SEQ ID NO: 17 - DNA, 19 bp, Artificial Sequence
  Description of Artificial Sequence Primer
agttgctctgcctctccac

SEQ ID NO: 18 - DNA, 20 bp, Artificial Sequence
  Description of Artificial Sequence Primer
cgtgattaaaaagcaccacc

SEQ ID NO: 19 - DNA, 126 bp, Artificial Sequence
  Description of Artificial Sequence Mut E1A promoter sequence
catcatcaataatataccttattttggattgaagccaatatgataatgaggtggtggccctttgatcttaatccctttgatctggatccctttgatctccaaccctttgatctagtcctatttata

SEQ ID NO: 20 - DNA, 9 bp, Artificial Sequence
  Description of Artificial Sequence Promoter replacement sequence
atcaaaggg

SEQ ID NO: 21 - DNA, 23 bp, Artificial Sequence
  Description of Artificial Sequence Promoter replacement sequence
atcaaagggatccagatcaaagg

SEQ ID NO: 22 - DNA, 52 bp, Artificial Sequence
  Description of Artificial Sequence Promoter replacement sequence
atcaagggttggagatcaaagggatccagatcaaagggattaagatcaaagg

SEQ ID NO: 23 - DNA, 53 bp, Artificial Sequence
  Description of Artificial Sequence Promoter replacement sequence
atcaaagggttggagatcaaagggatccagatcaaagggattaagatcaaagg

SEQ ID NO: 24 - DNA, 654 bp, Escherichia coli
atggatatcatttctgtcgccttaaagcgtcattccactaaggcatttgatgccagcaaaaaacttaccccggaacaggccgagcagatcaaaacgctactgcaatacagcccatccagcaccaactcccagccgtggcattttattgttgccagcacggaagaaggtaaagcgcgtgttgccaaatccgctgccggtaattacgtgttcaacgagcgtaaaatgcttgatgcctcgcacgtcgtggtgttctgtgcaaaaaccgcgatggacgatgtctggctgaagctggttgttgaccaggaagatgccgatggccgctttgccacgccggaagcgaaagccgcgaacgataaaggtcgcaagttcttcgctgatatgcaccgtaaagatctgcatgatgatgcagagtggatggcaaaacaggtttatctcaacgtcggtaacttcctgctcggcgtggcggctctgggtctggacgcggtacccatcgaaggttttgacgccgccatcctcgatgcagaatttggtctgaaagagaaaggctacaccagtctggtggttgttccggtaggtcatcacagcgttgaagattttaacgctacgctgccgaaatctcgtctgccgcaaaacatcaccttaaccgaagtgtaa

SEQ ID NO: 25 - DNA, 477 bp, Saccharomyces cerevisiae
atggtgacagggggaatggcaagcaagtgggatcagaagggtatggacattgcctatgaggaggcggccttaggttacaaagagggtggtgttcctattggcggatgtcttatcaataacaaagacggaagtgttctcggtcgtggtcacaacatgagatttcaaaagggatccgccacactacatggtgagatctccactttggaaaactgtgggagattagagggcaaagtgtacaaagataccactttgtatacgacgctgtctccatgcgacatgtgtacaggtgccatcatcatgtatggtattccacgctgtgttgtcggtgagaacgttaatttcaaaagtaagggcgagaaatatttacaaactagaggtcacgaggttgttgttgttgacgatgagaggtgtaaaaagatcatgaaacaatttatcgatgaaagacctcaggattggtttgaagatattggtgagtag

SEQ ID NO: 26 - DNA, 576 bp, Encephalomyocarditis virus
cgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 27 - DNA, 492 bp, Adenovirus type 5
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgtgtagtgtatttatacccggtgagttcctcaa

SEQ ID NO: 28 - DNA, 340 bp, Adenovirus type 5
gtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg

SEQ ID NO: 29 - DNA, 481 bp, Artificial Sequence
  Description of Artificial Sequence Mut E4 promoter sequence
gtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcacactagcaaaacacctgggcgagtctccacgtaaacggtcaaagtccccgcggccctagacaaatattacgcgctatgagtaacacaaaattattcagatttcacttcctcttattcagttttcccgcgaaaatggccaaatcttactcggttacgcccaaatttactactagtggagatcaaagggatccagatcaaagggattaagatcaaagggccaccacctcattatcatattggcttcaatccaaaataaggtatattattgatgatg

SEQ ID NO: 30 - DNA, 22 bp, Artificial Sequence
  Description of Artificial Sequence Primer
tgcattggtaccgtcatctcta

SEQ ID NO: 31 - DNA, 20 bp, Artificial Sequence
  Description of Artificial Sequence Primer
gttgctctgcctctccactt

SEQ ID NO: 32 - DNA, 41 bp, Artificial Sequence
  Description of Artificial Sequence Primer
cagatcaaagggattaagatcaaagggccattatgagcaag

SEQ ID NO: 33 - DNA, 43 bp, Artificial Sequence
  Description of Artificial Sequence Primer
gatccctttgatctccaaccctttgatctagtccttaagagtc

SEQ ID NO: 34 - DNA, 21 bp, Artificial Sequence
  Description of Artificial Sequence Primer
gggcgagtctccacgtaaacg

SEQ ID NO: 35 - DNA, 21 bp, Artificial Sequence
  Description of Artificial Sequence Primer
gggcaccagctcaatcagtca

SEQ ID NO: 36 - DNA, 38 bp, Artificial Sequence
  Description of Artificial Sequence Primer
cggaattcaagcttaattaacatcatcaataatatacc

SEQ ID NO: 37 - DNA, 31 bp, Artificial Sequence
  Description of Artificial Sequence Primer
gcggctagccaccatggagcgaagaaaccca

SEQ ID NO: 38 - DNA, 21 bp, Artificial Sequence
  Description of Artificial Sequence Primer
gccaccggtacaacattcatt

SEQ ID NO: 39 - DNA, 40 bp, Artificial Sequence
  Description of Artificial Sequence Primer
agctgggctctcttggtacaccagtgcagcgggccaacta

SEQ ID NO: 40 - DNA, 42 bp, Artificial Sequence
  Description of Artificial Sequence Primer
cccaccactgtagtgctgccaagagacgcccaggccgaagtt

SEQ ID NO: 41 - DNA, 36 bp, Artificial Sequence
  Description of Artificial Sequence Primer
ctgcgccccgctattggtcatctgaacttcggcctg

SEQ ID NO: 42 - DNA, 32 bp, Artificial Sequence
  Description of Artificial Sequence Primer
cttgcgggcggctttagacacagggtgcggtc

SEQ ID NO: 43 - DNA, 26 bp, Artificial Sequence
  Description of Artificial Sequence Primer
cagatcaaagggccattatgagcaag

SEQ ID NO: 44 - DNA, 28 bp, Artificial Sequence
  Description of Artificial Sequence Primer
gatccctttgatctagtccttaagagtc

SEQ ID NO: 45 - DNA, 21 bp, Artificial Sequence
  Description of Artificial Sequence Primer
atggcacaaactcctcaataa

SEQ ID NO: 46 - DNA, 22 bp, Artificial Sequence
  Description of Artificial Sequence Primer
ccaagactactcaacccgaata

SEQ ID NO: 47 - DNA, 143 bp, Artificial Sequence
  Description of Artificial Sequence Mut E1A promoter sequence
catcatcaataatataccttattttggattgaagccaatatgataatgaggtggtggccctttgatcttaatccctttgatctggatccctttgatctccaaccctttgatctagtcctatttatacccggtgagttcctcaa

SEQ ID NO: 48 - DNA, 24 bp, Artificial Sequence
  Description of Artificial Sequence Primer
agtttctttattcttgggcaatgt

SEQ ID NO: 49 - DNA, 23 bp, Artificial Sequence
  Description of Artificial Sequence Primer
agtcgtttgtgttatgtttcaac

SEQ ID NO: 50 - DNA, 60 bp, Artificial Sequence
  Description of Artificial Sequence Primer
tcgctagccaggcacaatcttcgcatttctttttttccagatggtgacagggggaatggc

SEQ ID NO: 51 - DNA, 28 bp, Artificial Sequence
  Description of Artificial Sequence Primer
tgactagttattcaccaatatcttcaaa

SEQ ID NO: 52 - DNA, 24 bp, Artificial Sequence
  Description of Artificial Sequence Primer
atgctagcgaattccgcccctctc

SEQ ID NO: 53 - DNA, 30 bp, Artificial Sequence
  Description of Artificial Sequence Primer
atactagttatgcatattatcatcgtgttt

SEQ ID NO: 54 - DNA, 35 bp, Artificial Sequence
  Description of Artificial Sequence Primer
ggaattcgctagtttctctactcttgggcaatgta

SEQ ID NO: 55 - DNA, 30 bp, Artificial Sequence
  Description of Artificial Sequence Primer
ggtggtggagatgctaaactcactttggtc

SEQ ID NO: 56 - DNA, 20 bp, Artificial Sequence
  Description of Artificial Sequence Primer
gtgacagggggaatggcaag

SEQ ID NO: 57 - DNA, 29 bp, Artificial Sequence
  Description of Artificial Sequence Primer
tgactagtttattcaccaatatcttcaaa

SEQ ID NO: 58 - DNA, 19 bp, Artificial Sequence
  Description of Artificial Sequence Primer
gccattaatgcaggagatg

SEQ ID NO: 59 - DNA, 20 bp, Artificial Sequence
  Description of Artificial Sequence Primer
ggagaaaggactgtgtactc

SEQ ID NO: 60 - DNA, 28 bp, Artificial Sequence
  Description of Artificial Sequence Primer
aggatccactctcttccgcatcgctgtc

SEQ ID NO: 61 - DNA, 23 bp, Artificial Sequence
  Description of Artificial Sequence Primer
agggttttcccagtcacgacgtt

SEQ ID NO: 62 - DNA, 24 bp, Artificial Sequence
  Description of Artificial Sequence Primer
agcggataacaatttcacacagga

SEQ ID NO: 63 - protein, 6 aa, Artificial Sequence
  Description of Artificial Sequence Illustrative peptide
EDPNEE

SEQ ID NO: 64 - protein, 6 aa, Artificial Sequence
  Description of Artificial Sequence Illustrative peptide
AAAAAG

SEQ ID NO: 65 - protein, 4 aa, Artificial Sequence
  Description of Artificial Sequence Illustrative peptide
LDLS

SEQ ID NO: 66 - protein, 4 aa, Artificial Sequence
  Description of Artificial Sequence Illustrative peptide
AAAA

(19) United States
(12) Patent Application Publication
(10) Pub. No.: US 2005/0175589 A1
(43) Pub. Date: Aug. 11, 2005

(54) Anti-neoplastic viral agents

(75) Inventors: Richard Derek Iggo, Lausanne (CH); Christophe Fuerer, Renens (CH); Krisztian Gyula Homicsko, Epalinges (CH)

Correspondence Address: NIXON & VANDERHYE, PC, 901 NORTH GLEBE ROAD, 11TH FLOOR, ARLINGTON, VA, 22203, US

(73) Assignee: BTG International Limited

(21) Appl. No.: 10/612,285

(22) Filed: Jul. 3, 2003

Related U.S. Application Data
(63) Continuation-in-part of application No. 10/433,681, filed on Dec. 23, 2003.

(30) Foreign Application Priority Data
Jul. 13, 2001 (GB) 0117198.2
Jul. 12, 2002 (WO) PCT/GB02/03211

(51) Int. Cl. A61K 48/00; C12N 15/861; C12N 7/00

(52) U.S. Cl. 424/93.2; 435/235.1; 435/456

(57) ABSTRACT

A viral DNA construct, and virus encoded thereby, is provided having one or more tumor specific transcription factor binding sites in place of one or more wild type transcription factor binding sites operatively positioned in the promoter region which controls expression of E1A open reading frame in the presence of said selected transcription factor, wherein the level or activity of the transcription factor is increased in a human or animal tumor cell relative to that of a normal human or animal cell of the same type; and wherein the viral DNA construct further comprises a therapeutic gene. Compositions and constructs contained therein are provided, particularly for use in therapy. Methods of treating patients for neoplasms are also provided.

The present invention provides viral agents that have application in the treatment of neoplasms such as tumors, particularly tumors derived from colon cells, more particularly liver tumors that are metastases of colon cell primary tumors. Still more particularly are provided replication competant, and particularly replication efficient, adenovirus constructs that selectively replicate in response to transcription activators present in tumor cells, these factors being present either exclusively or at elevated levels in tumor cells as compared to other cells, and thus which lead to tumor cell death and cell lysis.

By injecting the viral agents of the invention locally into the liver it is possible to treat liver metastases, which are a major cause of morbidity in colon cancer patients. Applications beyond this, e.g. to other sites and other tumors, such as colorectal cancers and melanomas, are also provided.

Viruses which replicate selectively in tumor cells have great potential for gene therapy for cancer as they can spread progressively through a tumor until all of its cells are destroyed. This overcomes the need to infect all tumor cells at the time the virus is injected, which is a major limitation to conventional replacement gene therapy, because in principle virus goes on being produced, lysing cells on release of new virus, until no tumor cells remain. An important fundamental distinction in cancer gene therapy is thus between single hit approaches, using non-replicating viruses, and multiple hit approaches, using replicating viruses.

In practice, only a few cycles of reinfection with the virus can occur before the immune system halts the infection. Even a single cycle of infection should lead to a massive local increase in virus concentration within the tumor, making it possible to achieve the same level of infection of tumor cells after injecting much smaller amounts of replicating than non-replicating viruses. Since the toxicity of adenoviruses is closely linked to the amount of virus injected, the risk of immediate life threatening reactions is potentially much lower with replicating viruses.

The prototype tumor selective virus is a defective adenovirus lacking the E1B 55K gene (dl 1520/ONYX 015, Bischoff et al., 1996). In normal adenoviruses 55K inactivates p53, hence it should not be required in cells where p53 is mutant. In practice, many cells containing wild type p53 are killed by the virus (Heise et al., 1997). The present inventors have tested this in H1299 p53-null lung carcinoma cells containing wild type p53 under a tetracycline-regulated promoter and found that d1 1520 replicates as well in the presence as in the absence of wild type p53. Besides targeting p53, E1B 55K is required for selective viral RNA export (Shenk, 1996) and it is not immediately obvious how loss of p53 could substitute for this function. At present there is no convincing evidence that dl 1520 targets p53 defects (Goodrum 1997, Goodrum 1998, Hall 1998, Rothman 1998, Turnell 1999).

As with p53-expressing viruses, combination therapy with chemotherapy and dl 1520 gives better results both in vitro and in xenografts (Heise et al., 1997). In principle, the virus should undergo multiple rounds of replication until there are no tumor cells remaining and since each infected cell produces 10³ to 10⁴ new virus particles, the amount of input virus should not be limiting. In practice, the required amount of dl 1520 virus injected is comparable for therapy with Ad-CMV-p53, a p53 supplementing virus. This means that the virus is not performing as expected for a replicating virus with the reasons for this again probably quite complex.

It is also possible to target early gene expression defects, as regulated by E2F, but this is complicated by the fact that as part of its life cycle the adenovirus already produces proteins (E1A and E4 orf 6/7) which target E2F. Since E1A and orf 6/7 are multifunctional proteins the effect of E1A and orf 6/7 mutations is complex and unpredictable.

In addition to E2F and p53, there are four transcription factors whose activity is known to increase in tumors. They are Tcf4, RBPJκ and Gli-1, representing the endpoints of the wnt, notch and hedgehog signal transduction pathways (Dahmane et al., 1997; Jarriault et al., 1995; van de Wetering et al., 1997) and HIF1alpha, which is stabilised by mutations in the Von Hippel Lindau tumor suppressor gene (Maxwell et al 1999). Mutations in APC or β-catenin are universal defects in colon cancer (Korinek et al., 1997; Morin et al., 1997); but they also occur at lower frequency in other tumors, such as melanoma (Rubinfeld et al., 1997). Such mutations lead to increased Tcf activity in affected cells. The hedgehog pathway is activated by mutations in the patched and smoothened proteins in basal cell cancer (Stone et al., 1996; Xie et al., 1998). Notch mutations occur in some leukaemias (Ellisen et al., 1991). Telomerase activation is one of the hallmarks of cancer (Hanahan D. and Weinberg R A. The hallmarks of cancer. Cell. 100, 57-70, 2000) and results from increased activity of the telomerase promoter, although the mechanism is unknown. According to Cong Y S et al (1999, HMG 8, 137-42) the elements responsible for promoter activity are contained within a region extending from 330 bp upstream of the ATG to the second exon of the gene and thus this sequence is a further suitable promoter sequence for use in the viral constructs and viruses of the invention.

Copending WO 00/56909, incorporated herein by reference, describes adenoviruses that replicate in response to activation of tumor specific transcription factors, particularly of the wnt signalling pathway. Wnt signalling is pathologically activated in virtually all colon tumors and this leads to transcription from promoters containing Tcf binding sites. The constitutive activation of the wnt pathway is caused by mutations in the APC, axin and β-catenin genes, thus inhibiting GSK-3B phosphorylation of β-catenin and its subsequent degradation by the proteasome (34). Cytoplasmic β-catenin enters the nucleus, where it can associate with members of the Tcf/Lef family of transcription factors and activate transcription of wnt target genes, such as c-myc, cyclin D1, Tcf1 and matrilysin.

WO/00/56909 describes a viral construct in which Tcf binding sites are placed in the adenovirus E2 promoter, which regulates expression of the viral replication genes. Mutations elsewhere in the virus or cell cannot bypass the absolute requirement for E2 gene products in viral replication. In order to achieve tight regulation of E2 transcription, the adjacent E3 enhancer was also mutated. Tcf sites were also placed in the E1B promoter, although the level of regulation achieved did not affect viral replication in vitro. These “Tcf” viruses showed a 50 to 100-fold decrease in replication in non-permissive cell lines whereas their activity was comparable to wild type Ad5 in many colon cancer cell lines.

The present inventors have now found that some colon cell lines are only semi-permissive for the tumor specific viruses of WO 00/56909, making it desirable to alter the viral genome of these constructs to increase their breadth of effective activity to include these cells. Such broadening will also be calculable to increase efficacy against other tumors where the Tcf pathway is implicated, eg. such as hepatocellular carcinoma and some breast, B cell, T cell, pancreatic, endometrial and ovarian cancers.

The present inventors have tested two different approaches to generate such viruses active in a broader range of colon cell lines: (i) insertion of tumor specific sites (eg. Tcf sites as described above) in the E1A promoter region, and (ii) mutation of the p300 binding site in E1A. The wild type E1A enhancer contains two types of regulatory element, termed I and II, which overlap the packaging signal (See FIG. 1). In addition to elements I and II, there are transcription factor binding sites in the inverted terminal repeat (ITR) and close to the E1A TATA box.

The amino-terminus of E1A contains a region of E1A that binds p300, a histone acetylase which functions as a general transcription factor. E1A activates promoters that contain ATF sites. WO 00/56909 virus vMB13 retains the ATF site in the E3 promoter providing advantage in this respect. The problem is that if a promoter does not have an ATF site, E1A will repress it by binding p300. For example: E1A blocks p53-dependent transcription in a manner that requires the p300 binding site in E1A. Tcf repression by E1A is a possibility in some cell lines, so mutation of the E1A p300-binding site may be preferred for such treatment where Tcf is used for cellular targeting.

The present inventors see a difference between the previously disclosed vMB13 and vMB14 in HCT116 cells, where the only difference between the two viruses is in the ATF site in the E3 promoter. Thus mutation of the E1A p300-binding site in vMB14 might be advantageous. Alternatively, the difference could be due to direct activation of the ATF site because Xu L et al (2000, Genes Dev 14, 585-595) report that ATF/CREB sites can be activated by wnt signals, although the mechanism is unknown.

Thus in a first aspect of the present invention there is provided a viral DNA construct encoding for an adenovirus capable of replication in a human or animal tumor cell, and preferably causing death of such tumor cells, characterised in that it comprises one or more selected transcription factor binding sites operatively positioned together with the E1A open reading frame such as to promote expression of E1A proteins in the presence of said selected transcription factor, the level or activity of which factor being increased in a human or animal tumor cell relative to that of a normal human or animal cell of the same type, i.e. lacking said transcription binding sites. Preferably the viral construct encodes for a virus that will cause death of the tumor cell directly, but in other embodiments it may encode a protein such as a vaccine, with the virus advantageously acting as adjuvant.

Preferably the viral DNA construct has a nucleic acid sequence corresponding to that of a wild type virus sequence characterised in that it has all or part of the wild type E1A transcription factor binding site replaced by the one or more selected transcription factor binding sites. More preferably the wild type E1A enhancer is deleted from its usual location or inactivated eg by mutation.

For the purposes of maintaining packaging capability of the construct the wild type packaging signal is preferably deleted from its wild type position (near the left hand inverted terminal repeat (ITR) in Ad5) and inserted elsewhere in the construct, in either orientation. Preferably the packaging signal is inserted adjacent the right hand terminal repeat, preferably within 600 bp of said ITR.

Preferably the E4 promoter contains the part of the E1A enhancer of the packaging signal flanked by Tcf and E4F sites.

Still more preferably one or more of the selected transcription factor binding sites are inserted into the right hand terminal repeat such as to provide sufficient symmetry to allow it to base pair to the left hand ITR during replication.

It will be realised from WO/00/56909 that the selected transcription factor binding sites are advantageously for a transcription factor whose activity or level is specifically increased by causal oncogenic mutations.

Preferably the nucleic acid sequence corresponds to that of the genome of an adenovirus with the selected transcription factor binding sites operatively positioned to control expression of the respective E1A genes. As with the viruses of WO 00/56909, the construct may advantageously have its nucleic acid sequence, other than the selected sites, corresponding to that of the genome of adenovirus Ad5, Ad40 or Ad41, or incorporates DNA encoding for fibre protein from Ad 5, Ad40 or Ad41, optionally with 1 to 30, more preferably 5 to 25, eg 15 to 25 lysines added to the end thereof.

Preferred constructs encode a functional viral RNA export capacity, eg. they have an E1 region wherein the E1B 55K gene is functional and/or intact.

The preferred tumor specific transcription factor binding site used in place of wild type site is selected from Tcf-4, RBPJκ, Gli-1, HIF1alpha and telomerase promoter binding sites. Preferred transcription factor binding sites are selectively activated in tumor cells containing oncogenic APC and β-catenin mutations. eg. the replacement sites are single or multiples of a Tcf-4 binding site sequence. eg. comprising from 2 to 20 Tcf-4 binding site sequences at each replaced promoter site.

In addition to the essential substitution of control of E1A orf, one or more of the more selected transcription factor binding sites may also be operatively positioned together with one or more of the E1B, E2 and E3 open reading frame such as to promote expression of the E1B, E2 and E3 proteins in the presence of said selected transcription factor. Also preferably are mutations in one or more residues in the NF1, NFκB, AP1 and ATF regions of the E3 promoter. Preferably the E2 late promoter is also inactivated with silent mutations.

Viruses comprising or encoded by the DNA constructs described above are also provided.

In a further aspect is provided a viral DNA construct, or a virus, of the invention for use in therapy, particularly therapy of patients having neoplasms.

In a still further aspect is provided a viral DNA construct, or a virus, of the invention in the manufacture of a medicament for the treatment of neoplasms.

In a still further aspect of the present invention is provided a therapeutic composition comprising a viral construct, or a virus, of the invention together with a physiologically acceptable carrier. Particularly compositions are characterised in that they are sterile and pyrogen free with the exception of the presence of the viral construct or virus encoded thereby. For example the carrier may be a physiologically acceptable saline.

In a still further aspect is provided a method of manufacture of a viral DNA construct or a virus encoded thereby, as provided by the invention characterised in that it comprises transforming an adenovirus viral genome having one or more wild type transcription factor binding sites controlling transcription of E1A, and optionally E4 open reading frames, such as to replace one or more of these by tumor specific transcription factor binding sites. Preferred methods clone the viral genome by gap repair in a circular YAC/BAC in yeast. Preferably the genome is modified by gap repair into a mutant vector for modification of sequences near the ITRs or by two step gene replacement for modification of internal sequences. For example the modified genome may be transferred to a prokaryote for production of viral construct DNA. Preferably the genome is transferred to a mammalian cell for production of virus.

In a still further aspect of the present invention there is provided a method for treating a patient suffering from a neoplasm wherein a viral DNA construct or virus of the invention is caused to infect tissues of the patient, including or restricted to those of the neoplasm, and allowed to replicate such that neoplasm cells are caused to be killed.

To produce a tightly regulated tumor specific transcription factor driven virus, a mutant E1A promoter, such as a Tcf-E1A promoter, needs to be installed. To effect this the present inventors have substituted part of the left hand inverted terminal repeat (ITR) of the virus with tumor specific promoter, e.g. Tcf binding sites. More preferably the E1A enhancer is deleted from its wild type location, in part or in full, more preferably completely. Most preferably the packaging signal is relocated from its wild type site near the left hand ITR to another part of the viral genome where it is still effective to allow packaging of the virus. This is preferably relocated to adjacent the right hand ITR, more preferably to within 600 bp thereof. The packaging signal may be relocated in either orientation.

The tumor transcription factor specific promoter conveniently comprises one or more Tcf binding sites, more preferably two to ten, still more preferably three to five Tcf sites in tandem. Most preferably four Tcf binding sites replace a portion of the ITR, the E1A enhancer and the packaging signal on the left hand side while the packaging signal sequence is introduced adjacent the right hand ITR to permit proper encapsidation of viral DNA.

The right side substitutions are particularly desirable to maintain the symmetry of the terminal repeats, so a similar or identical number of tumor specific transcription factor binding sites are inserted in the right ITR as provided in the left ITR, such as to allow these sites to become base paired together during replication. It will be realised that these insertions are preferably subsitutions as with the left side changes.

Tumor specific promoter-dependent transcription, eg with Tcf sites, is inhibited by E1A, so the inventors also investigated mutations in the E1A protein that would abolish this repression in transcription assays. Mutation of the p300 binding site in E1A partially relieved the repression, but in the context of the virus this mutation did not lead to increased transcription from the Tcf-E2 promoter and actually reduced the activity of the virus. Similar attenuation by mutation of the amino-terminus of E1A has been reported by the Onyx group. In contrast, it has now been surpisingly determined that the viruses containing only the transcription factor binding site changes in the E1A and E4 promoters (see for example vCF11 in the Examples herein) are selective for cells with active wnt signalling and active in most of the colon cancer cells studied.

Preferably the viruses of the invention also include tumor specific transcription factor binding sites in the promoter of the E2 open reading frame and more preferably also the promoter of the E3 open reading frame, as described in the copending patent WO 00/56909, which is incorporated herein by reference.

The Tcf sites in the preferred viruses of the present invention are adjacent to the TATA box in the Tcf-E1A promoter, but several hundred base pairs upstream of the E4 TATA box. To create an E1A promoter with the minimum possibility of interference from extraneous signals, all of the normal E1A regulatory elements were deleted from their wild type positions in a preferred construct and virus of the invention, vCF11.

This strategy contrasts with prior art approaches used to produce prostate, hepatocellular cancer and breast cancer targeting viruses, which retain the complete E1A enhancer but place exogenous promoters between it and the E1A start site. To remove the E1A enhancer in vCF11 it was necessary to transfer the viral packaging signal to the right ITR. In addition, approximately half of the right hand ITR was replaced by Tcf sites. This construction dictated the position of the Tcf sites relative to the E4 start site.

To optimise the Tcf-E4 promoter, it would be possible either to insert additional Tcf sites nearer the E4 start site or to delete the endogenous E4 control elements. The latter were retained in vCF11 because they confer repression of E4 transcription in normal cells. The mutant E4 promoter thus contains the part of the E1A enhancer contained in the packaging signal, which could activate the promoter, flanked by Tcf and E4F sites, which should repress the promoter in normal cells. The net result of these changes is reduced E4 transcription measured by luciferase assay, regardless of cell type.

Replication of the previous generation of viruses of WO 00/56909 is directed mainly at cells with activated wnt signalling by the Tcf sites in E2 promoter. The present invention viruses vCF22, 62 and 81, which have Tcf sites in multiple early promoters, are very selective but are relatively attenuated. The reduced activity in cytopathic effect assays seen with the viruses bearing mutations in all the early promoters might be due to deletion of element II in the E1A enhancer, which was previously reported to activate transcription of all early units in cis.

Comparison of different viruses shows that the Tcf-E1A promoter and Tcf-E2 promoters display the same hierarchy of activity in a panel of colon cell lines, but relative to the corresponding wild type promoters, the Tcf-E1A promoter is more active than the Tcf-E2 promoter. This probably explains why vCF11 is able to replicate better than vMB19 (see WO 00/56909) in Co115 cells.

To produce viruses that have substantially full spectrum activity using Tcf regulation of multiple early promoters is desirable to construct a Tcf-E2 promoter with much higher activity in the semi-permissive colon cells. Possible differences which could explain the reduced Tcf activity in some cell lines include increased expression of corepressors like groucho and CtBP, decreased expression of coactivators like p300 and CBP, pygopus, Bcl 9, acetylation or phosphorylation of Tcf4 preventing β-catenin binding or DNA binding, and increased activity of the ΔN-Tcf1 negative feedback loop.

Luciferase reporter assays show a systematic inhibition of Tcf-dependent transcription by E1A. Mutagenesis of E1A indicated that this effect was partly due to inhibition of p300 by E1A, consistent with reports that p300 is a coactivator for β-catenin. Coexpression of p300 together with E1A had the same effect on Tcf-dependent transcription as deletion of the p300 binding site in E1A, indicating that the remaining repression was unlikely to be due to inhibition of p300. The residual repressive effect of E1A could not be mapped to any known domain and merits further study. The negative results obtained with the ΔCR1 mutant are surprising because deletion of the CR1 p300-binding subdomain alone did partially restore Tcf-dependent transcription. This could conceivably be explained by an artefactual elevation of transcription of the renilla luciferase control by ΔCR1 E1A, but a more likely explanation is that another function of E1A is impaired by deletion of the entire CR1 domain.

The inhibition of Tcf-dependent transcription by E1A in the first generation viruses was greatest in the semi-permissive cell lines like Co115, resulting in very low luciferase activity because the starting level of Tcf activity was also lower in these cells. Hence, we expected to see a substantial effect of the Δ2-11 E1A mutation in the context of the viruses. In practice, the mutation produced no increase in expression from the Tcf promoters in colon cell lines and reduced the activity of the virus in cytopathic effect assays. The mutation had complex and inconsistent effects in burst assays: it appeared to reduce burst size in permissive cells when the E2 promoter was driven by E1A (ie wild type), but increase burst size in some non-permissive cells when the E2 promoter was driven by Tcf. A general explanation is that any gain in Tcf activity due to this E1A mutation was offset by a loss of other E1A activities. Since we only tested 12S E1A, it is possible that these functions map to the other E1A isoforms expressed during viral infection. In addition, there are some basal promoter activities regulated by E1A which may be abrogated by the Δ2-11 mutation.

The most mutant virus investigated, vCF62, lacks many of the transcriptional response elements through which E1A normally controls the virus (ATF sites in the E1A, E2, E3 and E4 promoters; E2F sites in the E2 promoter), and showed very large decreases in activity in semi-permissive cells in both burst and cytopathic effect assays.

Preferably the viral DNA construct is characterised in that it encodes a functional viral RNA export capacity. For adenovirus this is encoded in the E1 and E4 regions, particularly the E1B 55K and E4 orf 6 genes. Thus preferably the encoded virus is of wild type with respect to expression of these genes in tumor cells. Most preferably the E1B 55K and E4 orf 6 open reading frames are functional and/or intact where present in the corresponding wild type virus.

Preferred colon tumor specific adenoviruses are encoded by viral DNA constructs corresponding to the DNA sequence of Ad5 or one or more of the enteric adenoviruses Ad40 and Ad41 modified as described above. Ad40 and Ad41, which are available from ATCC, are selective for colon cells and one important difference to Ad5 is that there is an additional fibre protein. The fibre protein binds to the cell target host surface receptor, called the coxsackie-adeno receptor or CAR for Ad5. Colon cells have less CAR than lung cells which Ad5 is adapted to infect. Ad40 and Ad41 have two fibre proteins, with the possibility being that they may use two different receptors. The expected form of resistance to virus therapy is loss of the receptor, which obviously prevents infection. Genetic instability in tumors means this will happen at some reasonable frequency; about 1 in 100 million cells, a mutation rate of 1 in 108. If you delete two receptors you multiply the probabilities; ie. loss of both will occur in 1 in 1016 cells. A tumor contains between 109 and 1012 cells. Hence resistance is less likely to develop if a virus uses more than one receptor. One fibre protein in Ad40 and 41 uses CAR whilst the receptor used by the other is as yet unknown.

Advantageously the use of the constructs of the invention, particularly in the form of viruses encoded thereby, to treat neoplasms such as liver metastasis is relatively non-toxic compared to chemotherapy, providing good spread of virus within the liver aided by effective replication.

Preferred tumor specific transcription factor binding sites that are used in place of wild type sites are those described above as Tcf-4, HIF1alpha, RBPJκ and Gli-1 sites, and a fragment of the telomerase promoter conferring tumor-specific transcription.

A most preferred transcription factor binding site is that which binds Tcf-4, such as described by Vogelstein et al in U.S. Pat. No. 5,851,775 and is responsive to the heterodimeric β-catenin/Tcf-4 transcription factor. As such the transcription factor binding site increases transcription of genes in response to increased β-catenin levels caused by APC or β-catenin mutations. The telomerase promoter is described by Wu KJ. et al (1999, Nat Genet 21, 220-4) and Cong Y S. et al (1999 HumMol Genet 8, 137-42). A further preferred binding site is that of HIF1alpha, as described by Maxwell P H. et al, (1999 Nature 399, 271-5). One may use a HiF1alpha-regulated virus to target the hypoxic regions of tumors, involving no mutation of the pathway as this is the normal physiological response to hypoxia, or the same virus may be used to target cells with VHL mutations either in the familial VHL cancer syndrome, or in sporadic renal cell carcinomas, which also have VHL mutations. A retrovirus using the HIF promoter to target hypoxia in ischemia has already been described by Boast K. et al (1999 Hum Gene Ther 10, 2197-208).

Particularly the inventors have now provided viral DNA constructs, and viruses encoded thereby, which contain the Tcf transcription factor binding sites referred to above in operational relationship with the E1A, and optionally E4, open reading frames described above, particularly in place of wild type transcription factor binding sites in their promoters and shown that these are selective for tumor cells containing oncogenic APC and β-catenin mutations. Tcf-4 and its heterodimer bind to a site designated Tcf herein. Preferred such replacement sites are single or multiples of the Tcf binding sequence, eg. containing 2 to 20, more preferably 2 to 6, most conveniently, 2, 3 or 4 Tcf sites.

Particular Tcf sites are of consensus sequence (A/T)(A/T)CAA(A/T)GG, see Roose, J., and Clevers, H. (1999 Biochim Biophys Acta 1424, M23-37), but are more preferably as shown in the examples herein.

A preferred group of viral constructs and viruses of the invention are those having the further selected transcription factor binding site in a function relationship with the E2 orfs and more preferably also with the E3 orfs. Preferably the VIII region containing the E3 promoter is characterised in that it has mutations to one or more residues in the NF1, NFκB, AP1 and/or ATF regions of the E3 promoter, more preferably those mutations which reduce E2 gene transcription caused by E3 promoter activity. The present inventors have particularly provided silent mutations, these being such as not to alter the predicted protein sequence of any viral protein but which alter the activity of key viral promoters.

NFκB is strongly induced in regenerating liver cells, ie. hepatocytes (see Brenner et al J. Clin. Invest. 101 p 802-811). Liver regeneration to fill the space vacated by the tumor is likely to occur following successful treatment of metastases. In addition, if one wishes to treat hepatoma, which arise on a background of dividing normal liver cells, then destroying the NFκB site is potentially advantageous.

E1A normally activates the E2 promoter through the ATF site. In the absence of such targeting E1A represses promoters, eg. by chelating p300/CBP. When the ATF site is deleted in a mutant E2 promoter, E1A produced by the virus should reduce general leakiness of the mutant E2 promoter in all cell types. The E3 promoter is back-to-back with the E2 promoter and the distinction between them is defined but functionally arbitrary. Hence further reduction of the activity of the mutant E2 promoter is possible by modifying or deleting transcription factor binding sites in the E3-promoter. Since the E3 promoter lies in coding sequence it cannot just be deleted. Instead the inventors have provided up to 16 silent substitutions changing critical residues in known NF1, NFκB, AP1 and ATF sites (Hurst and Jones, 1987, Genes Dev 1, 1132-46, incorporated herein by reference).

Further viral constructs of the present invention may be provided by modifying the E2-late promoter of adenoviruses. The E2-early promoter controls transcription of DNA polymerase (pol), DNA binding protein (DBP) and preterminal protein (pTP). By mutating the E2 late promoter it is possible to have a similar effect, ie. at least in part, to the E1B deletion because E1B deletion reduces export of DBP RNA expressed from the E2 late promoter. DBP is required stoichiometrically for DNA replication, so reducing DBP production in normal cells is desirable. Since the E2 late promoter lies in 100 k protein coding sequence it cannot just be deleted. Instead the inventors have determined that it can inactivated with silent mutations changing critical residues in known transcription factor binding sites.

Particular transcription factor binding sites in the E2 late promoter were identified by DNase I footprinting (marked I-IV in FIG. 4 herein; Goding et al, 1987, NAR 15, 7761-7780). The most important is a CCAAT box lying in footprint II. Mutation of this CCAAT box reduces E2 late promoter activity 100-fold in CAT assays (Bhat et al, 1987,EMBO J, 6,2045-2052). One such mutation changes the marked CCAAT box sequence GAC CAA TCC to GAT CAG TCC. (see FIG. 4 below). This is designed to abolish binding of CCAAT box binding factors without changing the 100 k protein sequence. Additional silent mutations in the other footprints can be used to reduce activity further An further preferred or additional mutation possible is to regulate expression of E1B transcription by mutating the E1B promoter. This has been shown to reduce virus replication using a virus in which a prostate-specific promoter was used to regulate E1B transcription (Yu, D. C., et al 1999 Cancer Research 59, 1498-504). A further advantage of regulating E1B 55K expression in a tumor-specific manner would be that the risk of inflammatory damage to normal tissue would be reduced (Ginsberg, H. S., et al 199 PNAS 96, 10409-11). The inventors have produced viruses with Tcf sites replacing the E1B promoter Sp1 site to test this proposition.

It will be apparent to those skilled in the art that the viral constructs and viruses of the invention may optionally include a therapeutic gene. The inclusion of such a gene may be useful to enhance tumor cell killing.

Therapeutic genes are DNA sequences which encode a therapeutic protein or therapeutically active fragment thereof. Therapeutic proteins are proteins which have a therapeutically beneficial effect, such as those regulating the cell cycle, or inducing cell death. Examples of therapeutic proteins which regulate the cell cycle include p53, pRb and mitosin whereas genes which induce cell death includes toxins or conditional suicide genes e.g. thymidine kinase, nitroreductase, and cytosine deaminase. Cytokines which augment the immunological functions of effector cells may also be appropriate. Therapeutic genes are essentially heterologous genes, i.e. transgenes, which are expressed from the constructs and viruses of the invention.

In contrast with, for example, the Calydon viruses, the design of the present inventors viruses means that, despite retaining a full complement of adenoviral genes, spare packaging capacity is available, which can be used to express conditional toxins, such as the prodrug-activating enzyme HSV thymidine kinase (tk), nitroreductase (eg. from E. coli—see Sequence listing), cytosine deaminase (eg from yeast-m see Sequence listing), or other therapeutic protein. This could be expressed for example from the E3 promoter, whose activity is regulated in some of the viruses, to provide an additional level of tumor targeting. Alternatively, it could be expressed from a constitutive promoter to act as a safety feature, since ganciclovir would then be able to kill the virus. Constitutive tk expression in an E1B-deficient virus also increases the tumor killing effect, albeit at the expense of replication (Wildner, O., et al 1999 Gene Therapy 6, 57-62). An alternative prodrug-activating enzyme to express would be cytosine deaminase (Crystal, R. G., et al 1997 Hum Gene Ther 8, 985-1001), which converts 5FC to 5FU. This has advantage because 5FU is one of the few drugs active on liver metastases, the intended therapeutic target, but produces biliary sclerosis in some patients. In a preferred virus the ‘suicide gene’ e.g. sequence encoding the toxin, is expressed from a position between the fiber and the E4 region. This gene is preferably and expressed late either with an IRES or by differential splicing, that is, in a replication-dependant manner. Such aspect is novel and inventive in its own right and forms an independent invention.

Late expression of a therapeutic gene, e.g. a toxin or suicide gene, may preferable to early expression, as early expression risks killing the virus too soon, e.g. if the toxin interferes with viral DNA replication, and also requires effective expression from a single copy of the viral genome. Late expression of therapeutic genes is also attractive because replication can increase the number of transcription templates to many thousands of copies. Provided viral replication is restricted to tumor cells, e.g. by insertion of tumor specific transcription factor binding sites in the early viral promoters, therapeutic genes expressed from late promoters should also be restricted to tumor cells, so there is no a priori reason to use a tumor specific promoter to control expression of the toxin or suicide gene.

There are two possible strategies which may be used to express a therapeutic gene in a virus of the invention: addition or replacement. Replacement of a viral gene involves the deletion of a viral gene which is then replaced with a therapeutic gene. This approach carries the risk that the virus may be less active in-vivo. Addition of a therapeutic gene, either as a complete new transcription unit or as a new open reading frame in an existing transcription unit is preferred. Addition of a new transcription unit is typically used in non-replicating viruses, for example, by inserting a CMV driven tk gene in the E1 region. Given the adenovirus packaging size limit this is very difficult to achieve with a replicating virus. However, the present inventors have been able to produce a viral DNA construct which retains a full complement of adenoviral genes, and which has the spare packaging capacity available to allow addition of a therapeutic gene. By removing regions from the wild type virus promoters, which are larger than the tumor specific transcription factor binding sites that are inserted in their place, the inventors have been able reduce the overall size of the viral construct, thus providing sufficient space in the viral genome to allow the insertion of a therapeutic gene.

It will be apparent to those skilled in the art that although the constructs and viruses of the present invention provide spare packaging capacity, the size of the therapeutic gene which may be inserted, by addition or replacement, is limited by the adenovirus packaging limit, which was reported to be 105% of wild type genome size, Bett A J, et al. J Virol 1993; 67: 5911-5921. Thus preferably the genome size of the viral constructs and viruses of the invention does not exceed 105%.

Thus a further aspect of the invention provides a viral DNA construct encoding for an adenovirus capable of replication in a human or animal tumor cell comprising one or more selected tumor specific transcription factor binding sites replacing one of more wild type transcription factor binding sites in the viral promoter sequences such as to control expression of viral genes, wherein the level or activity of the tumor specific transcription factor is increased in a human or animal tumor cell relative to that of a normal human or animal cell of the same type; and a therapeutic gene; and wherein the viral construct encodes a full complement of adenoviral proteins.

Preferably the therapeutic gene is a suicide gene, preferably positioned in the major late transcription unit, yet more preferably between the fiber gene and the E4 region, for example, immediately after the fiber gene.

Preferably the therapeutic, e.g. suicide gene, is expressed in a replication-dependent manner from the major late transcription unit.

A further aspect of the invention provides a method of producing a viral DNA construct encoding for an adenovirus capable of selective replication in a human or animal tumor cell comprising removal of regions comprising one or more wild type transcription factor binding sites from one or more viral promoters and replacement of said regions with one or more tumor specific transcription factor binding sites, wherein the replacement with tumor specific transcription factor provides spare packaging capacity in the viral construct; inserting a therapeutic gene; and retaining a full complement of adenoviral genes in the construct.

Expression of a therapeutic gene within an existing transcription unit is possible by making a fusion protein, by reinitiation of translation from an internal ribosome entry site (IRES) or by alternative splicing. Of the two methods tested for expressing yCD, the IRES gave higher expression. Expression of a therapeutic gene using an IRES in this site was recently demonstrated with p53, Sauthoff H et al. Hum Gene Ther 2002; 13: 1859-1871. One disadvantage of EMCV IRES is its relatively large size (588 bp). A recent study found that another IRES, eIF4G, which is only 339 bp long, gave substantially higher expression than EMCV; Wong et al. Gene Ther 2002; 9: 337-344.

Preferably the L5/E4 junction is used as the site for insertion of the therapeutic or suicide gene, e.g. yCD, as there is a progressive use of more distal splice sites in the major late transcript over the course of infection. Use of a putative L6 transcript would guarantee the maximum restriction of expression to cells that are committed to viral replication. This is desirable for a suicide gene whose expression is not restricted to tumor cells in any other way.

If the major concern is to avoid increasing the size of the virus while maintaining a full complement of viral genes, the most attractive ways to express a suicide gene are fusion to a viral protein or alternative splicing. Even if activity could be retained, fusion of e.g. yCD to the fibre protein would be unattractive, if only because the unique role of the fibre protein in viral tropism means it is likely to be heavily modified in other ways in any successful therapeutic virus. The complexity of splicing of the major late transcript appears to make alternative splicing an even less attractive option. However the inventors have now demonstrated the feasibility of inserting an additional L6splicaing unit in the major late transcript of Ad5. Thus in a preferred embodiment the therapeutic gene is expressed as an additional L6 splicing unit in the major late transcript.

A further preferred aspect of the invention provides a viral DNA construct encoding for an adenovirus capable of replication in a human or animal tumor cell comprising one or more selected transcription factor binding sites operatively positioned together with the E1A open reading frame such as to promote expression of E1A proteins in the presence of said selected transcription factor, wherein the level or activity of the transcription factor is increased in a human or animal tumor cell relative to that of a normal human or animal cell of the same type; and wherein the viral construct further comprises a therapeutic gene.

Preferably the therapeutic gene is a suicide gene positioned between the fibre gene and the E4 region in the major late transcription unit of the viral construct, preferably the suicide gene is selected from HSV thymidine kinase, nitroreductase and cytosine deaminase.

Preferably the therapeutic gene is expressed late in a replication-dependent manner using an IRES or by differential splicing. More preferably the viral construct of the invention include a therapeutic gene and retain a full complement of adenoviral genes.

Still more preferably the viral constructs or viruses including the therapeutic gene have the wild type packaging signal deleted from its wild type site adjacent the left hand inverted terminal repeat (ITR) and inserted elsewhere in the construct, in either forward or backward orientation.

Preferably the viral constructs or viruses including the therapeutic gene have the selected transcription factor binding sites operatively positioned together with the E1A open reading frame such as to promote expression of E1A proteins in the presence of the selected transcription factor; preferably the selected transcription factor binding site is a Tcf-4 transcription factor binding site.

Preferably the E4 promoter contains part of the E1A enhancer of the packaging signal flanked by Tcf and E4F sites.

In a still further aspect of the invention there is provided a viral DNA construct encoding for an adenovirus capable of replication in a human or animal tumor cell comprising one or more selected transcription factor binding sites operatively positioned together with one or more of the E1B, E2 and E3 open reading frames such as to promote expression of one or more E1B, E2 and E3 proteins in the presence of said selected transcription factor, wherein the level or activity of the transcription factor is increased in a human or animal tumor cell relative to that of a normal human or animal cell of the same type; and a therapeutic gene.

Preferably the selected transcription factor binding sites are selected from Tcf-4, RBPJκ, Gli-1, HIF1 alpha and telomerase promoter binding sites.

Preferably the therapeutic gene is a suicide gene expressed in a replication-dependent manner, preferably the therapeutic gene is positioned between the fibre gene and E4 in the major late transcription unit.

Preferably the selected transcription factor binding sites are inserted into the right hand terminal repeat such as to provide sufficient symmetry to allow it to base pair to the left hand ITR during replication.

Preferably the viral constructs and viruses of the invention encode a full complement of adenoviral proteins.

Having produced a virus with one or more levels of regulation to prevent or terminate replication in normal cells, it is further preferred and advantageous to improve the efficiency of infection at the level of receptor binding. The normal cellular receptor for adenovirus, CAR, is poorly expressed on some colon tumor cells. Addition of a number of lysine residues, eg 1 to 25, more preferably about 5 to 20, to the end of the adeno fibre protein (the natural CAR ligand) allows the virus to use heparin sulphate glycoproteins as receptor, resulting in more efficient infection of a much wider range of cells. This has been shown to increase the cytopathic effect and xenograft cure rate of E1B-deficient viruses (Shinoura, H., et al 1999 Cancer Res 59, 3411-3416 incorporated herein by reference). Fibre mutations that alter NGR, PRP or RGD targeting may also be expolited, eithre increasing or decreasing such effect depending upon the need to increase or decrease infectivity toward given cell types.

An alternative strategy is to incorporate the cDNA encoding for Ad40 and/or Ad41 fibres, or other efficaceous fibre type such as Ad3 and Ad35 into the construct of the invention as described above. The EMBL and Genbank databases list such sequences and they are further described in Kidd et al Virology (1989) 172(1), 134-144; Pieniazek et al Nucleic Acids Res. (1989) November 25 ;17-20, 9474; Davison et al J. Mol. Biol (1993) 234(4) 1308-16; Kidd et al Virology (1990) 179(1) p139-150; all of which are incorporated herein by reference.

In a second aspect of the invention there is provided the viral DNA construct of the invention, particularly in the form of a virus encoded thereby, for use in therapy, particularly in therapy of patients having neoplasms, eg. malignant tumors, particularly colorectal tumors and most particularly colorectal metastases. Most preferably the therapy is for liver tumors that are metastases of colorectal tumors.

In a third aspect there is provided the use of a viral DNA construct of the invention, particularly in the form of a virus encoded thereby, in the manufacture of a medicament for the treatment of neoplasms, eg. malignant tumors, particularly colorectal tumors and most particularly colorectal metastases. Most preferably the treatment is for liver tumors that are metastases of colorectal tumors.

In a fourth aspect of the invention there are provided compositions comprising the viral DNA construct of the invention, particularly in the form of a virus encoded thereby, together with a physiologically acceptable carrier. Such carrier is typically sterile and pyrogen free and thus the composition is sterile and pyrogen free with the exception of the presence of the viral construct component or its encoded virus. Typically the carrier will be a physiologically acceptable saline.

In a fifth aspect of the invention there is provided a method of manufacture of the viral DNA construct of the invention, particularly in the form of a virus encoded thereby comprising transforming a viral genomic DNA, particularly of an adenovirus, having wild type E1A transcription factor binding sites, particularly as defined for the first aspect, such as to operationally replace these sites by tumor specific transcription factor binding sites, particularly replacing them by Tcf transcription factor binding sites. Operational replacement may involve partial or complete deletion of the wild type site. Preferably the transformation inserts two or more, more preferably 3 or 4, Tcf-4 transcription factor binding sites. More preferably the transformation introduces additional mutations to one or more residues in the NF1, NFκB, AP1 and/or ATF binding sites in the E3 promoter region of the viral genome. Such mutations should preferably eliminate interference with E2 activity by E3 and reduce expression of E2 promoter-driven genes in normal cells and non-colon cells. Reciprocally, it preferably replaces normal regulation of E3 with regulation by Tcf bound to the nearby E2 promoter.

Traditional methods for modifying adenovirus require in vivo reconstitution of the viral genome by homologous recombination, followed by multiple rounds of plaque purification. The reason for this is the difficulty of manipulating the 36 kb adenovirus genome using traditional cloning techniques. Newer approaches have been developed which circumvent this problem, particularly for E1-replacement vectors. The Transgene and Vogelstein groups use gap repair in bacteria to modify the virus (Chartier et al., 1996; He et al., 1998). This requires the construction of large vectors which are specific for each region to be modified. Since these vectors are available for E1-replacement, these approaches are very attractive for construction of simple adenoviral expression vectors. Ketner developed a yeast-based system where the adenoviral genome is cloned in a YAC and modified by two step gene replacement (Ketner et al., 1994). The advantage of the YAC approach is that only very small pieces of viral DNA need ever be manipulated using conventional recombinant DNA techniques. Conveniently, a few hundred base pairs on either side of the region to be modified are provided and on one side there should be a unique restriction site, but since the plasmid is very small this is not a problem. The disadvantage of the Ketner approach is that the yield of YAC DNA is low.

The present inventors have combined the bacterial and yeast approaches which may contain mutant viral sequences. Specifically, they clone the viral genome by gap repair in a circular YAC/BAC in yeast, modify it by two step gene replacement, then transfer it to bacteria for production of large amounts of viral genomic DNA. The latter step is useful because it permits direct sequencing of the modified genome before it is converted into virus, and the efficiency of virus production is high because large amounts of genomic DNA are available. They use a BAC origin to avoid rearrangement of the viral genome in bacteria. Although this approach has more steps, it combines all of the advantages and none of the disadvantages of the pure bacterial or yeast techniques.

Although it can be used to make E1-replacement viruses, and the inventors have constructed YAC/BACs allowing cycloheximide selection of desired recombinants in the yeast excision step to simplify this task, the main strength of the approach is that it allows introduction of mutations at will throughout the viral genome. Further details of the YAC/BAC are provided by the inventors as their contribution to Gagnebin et al (1999) Gene Therapy 6, 1742-1750) which is incorporated herein by reference. Sequential modification at multiple different sites is also possible without having to handle large DNA intermediates in vitro.

The adenovirus strain to be mutated using the method of the invention is preferably a wild type adenovirus. Conveniently adenovirus 5 (Ad 5) is used, as is available from ATCC as VR5. The viral genome is preferably completely wild type outside the regions modified by the method, but may be used to deliver tumor specific toxic heterologous genes, eg. p53 or genes encoding prodrug-activating enzymes such as thymidine kinase which allows cell destruction by ganciclovir. However, the method is also conveniently applied using viral genomic DNA from adenovirus types with improved tissue tropisms (eg. Ad40 and Ad41).

In a sixth aspect of the present invention there is provided a method for treating a patient suffering from neoplasms wherein a viral DNA construct of the invention, particularly in the form of a virus encoded thereby, is caused to infect tissues of the patient, including or restricted to those of the neoplasm, and allowed to replicate such that neoplasm cells are caused to be killed.

The present invention further attempts to improve current intra-arterial hepatic chemotherapy by prior administration of a colon-targeting replicating adenovirus. DNA damaging and antimetabolic chemotherapy is known to sensitise tumor cells to another replicating adenovirus in animal models (Heise et al., 1997). For example, during the first cycle the present recombinant adenovirus can be administered alone, in order to determine toxicity and safety. For the second and subsequent cycles recombinant adenovirus can be administered with concomitant chemotherapy. Safety and efficacy is preferably evaluated and then compared to the first cycle response, the patient acting as his or her own control.

Route of administration may vary according to the patients needs and may be by any of the routes described for similar viruses such as described in U.S. Pat. No. 5,698,443 column 6, incorporated herein by reference. Suitable doses for replicating viruses of the invention are in theory capable of being very low. For example they may be of the order of from 10² to 10¹³, more preferably 10⁴ to 10¹¹, with multiplicities of infection generally in the range 0.001 to 100.

For treatment a hepatic artery catheter, eg a port-a-cath, is preferably implanted. This procedure is well established, and hepatic catheters are regularly placed for local hepatic chemotherapy for ocular melanoma and colon cancer patients. A baseline biopsy may be taken during surgery.

A typical therapy regime might comprise the following:

Cycle 1: adenovirus construct administration diluted in 100 ml saline through the hepatic artery catheter, on days 1, 2 and 3.

Cycle 2 (day 29): adenovirus construct administration on days 1, 2, and 3 with concomitant administration of FUDR 0.3 mg/kg/d as continuous infusion for 14 days, via a standard portable infusion pump (e.g. Pharmacia or Melody), repeated every 4 weeks.

Toxicity of viral agent, and thus suitable dose, may be determined by Standard phase I dose escalation of the viral inoculum in a cohort of three patients. If grade III/IV toxicity occurs in one patient, enrolment is continued at the current dose level for a total of six patients. Grade III/V toxicity in ≧50% of the patients determines dose limiting toxicity (DLT), and the dose level below is considered the maximally tolerated dose (MTD) and may be further explored in phase II trials.

It will be realised that GMP grade virus is used where regulatory approval is required.

It will be realised by those skilled in the art that the administration of therapeutic adenoviruses may be accompanied by inflammation and or other adverse immunological event which can be associated with eg. cytokine release. Some viruses according to the invention may also provoke this, particularly if E1B activity is not attenuated. It will further be realised that such viruses may have advantageous anti-tumor activity over at least some of those lacking this adverse effect. In this event it is appropriate that an immuno-suppressive, anti-inflammatory or otherwise anti-cytokine medication is administered in conjunction with the virus, eg, pre-, post- or during viral adminstration. Typical of such medicaments are steroids, eg, prednisolone or dexamethasone, or anti-TNF agents such as anti-TNF antibodies or soluble TNF receptor, with suitable dosage regimes being similar to those used in autoimmune therapies. For example, see doses of steroid given for treating rheumatoid arthritis (see WO93/07899) or multiple sclerosis (WO93/10817), both of which in so far as they have US equivalent applications are incorporated herein by reference.

In conclusion, we have shown that adenovirus replication can be regulated by insertion of Tcf sites into the E1A or E2 promoters. Mutation of the p300 binding site in E1A did not increase transcription from Tcf promoters in the context of the virus. Since the Δ2-11 mutation consistently reduced virus activity in cytopathic effect assays, it would be better to retain the p300 2-11 domain in therapeutic viruses.

To achieve strong activation of viral E2 transcription in cell lines with only weak Tcf activity will require the insertion of sites for synergistically acting transcription factors or modification of the basal promoter.

The present invention will now be described by way of illustration only by reference to the following non-limiting Examples, Methods, Sequences and Figures. Further embodiments falling within the scope of the claims will occur to those skilled in the art in the light of these.

TABLE 1
Structure of the adenoviruses used in this study

| virus | mutant | Promoters | | | | | ORF |
|---|---|---|---|---|---|---|---|
| name | regions^{a} | E1A | E1B | E2 | E3 | E4 | E1A |
| vCF11 | A4 | Tcf^{b} | wt | wt | wt | mut^{c} | wt |
| vCF42 | AΔ4 | Tcf | wt | wt | wt | mut | Δp300^{d} |
| vMB31 | B23′ | wt | Tcf | Tcf | mut + A^{e} | wt | wt |
| vCF22 | AB23′4 | Tcf | Tcf | Tcf | mut + A | mut | wt |
| vKH1 | AΔ4 | Tcf | Tcf | wt | wt | mut | wt |
| vMB19 | B23 | wt | Tcf | Tcf | mut − A^{f} | wt | wt |
| vCF81 | ΔB23 | wt | Tcf | Tcf | mut − A | wt | Δp300 |
| vCF62 | AΔB234 | Tcf | Tcf | Tcf | mut − A | mut | Δp300 |
| VCaK1 | ABFIS4 | Tcf | Tcf | wt | wt | mut | wt^{g} |

^{a}Abbreviations used in FIG 3.
^{b}Replacement of endogenous promoters by four Tcf binding sites.
^{c}Insertion of three Tcf binding sites and the packaging signal upstream of the endogenous promoter.
^{d}Deletion of amino acids 2-11 in E1A.
^{e}Mutation of the NF1, NFκB, and AP1 sites in the E3 promoter.
^{f}Mutation of the NF1, NFκB, AP1, and ATF sites in the E3 promoter.
^{g}Mutations of HSPG and CAR binding domain of fibre + insertion of RGD4c peptide in fibre H1 loop in CaK1 fibre + EMCV IRES driving translation of yeast cytosine deaminase from the late major transcript.

### FIGURES

FIG. 1.

(A) Schematic diagram showing the mutagenesis of the E1A promoter (upper part) and E4 promoter (lower part). Both regions are shown from the ITRs to the beginning of the first open reading frame. The dark triangles represent the A motifs in the packaging signal.

(B) Schematic diagram showing mutant regions in the viruses used in this study (see table 1 for details). To facilitate interpretation of the figures, the viruses are given clone names (vCFs and vMBs) and a codename summarising their structure: A, B, 2, 4=Tcf sites in the E1A, E1B, E2, and E4 promoters, respectively. 3=silent mutations in the NF1, NFκB, AP1, and ATF sites in the E3 promoter.3′=as 3, but without the ATF site mutation. Δ=deletion of amino acids 2-11 in E1A that abolishes p300 binding. F=mutations in the fibre that abolish HSPG and CAR binding together with insertion of an RGD4C peptide in the H1 loop. I=EMCV IRES. C=Yeast cytosine deaminase.

FIG. 2: Western blot of cMM1 cells probed for E1A and DBP 24 hours after infection with wild type AdS and Tcf-viruses. Tetracycline withdrawal leads to expression of ΔN-β-catenin (lanes 6-8). The Tcf-E1A promoter responds to activation of wnt signalling (lane 7).

FIG. 3. Western blot for E1A, E1B55k, DBP and E4orf6 24 hours after infection of different cell lines with wild-type AdS and Tcf viruses. SW480 and Isrec01 are permissive colon cancer cell lines. Co115, Hct116 and HT29 are semi-permissive colon cancer cell lines. H1299, HeLa and SAEC are non-permissive cell lines in which the wnt pathway is inactive. (The SAEC blot is derived from two separate experiments giving similar wild-type Ad5 activity. vMB31 was not tested on SAEC)

FIG. 4. Bar chart of results of luciferase assays in SW480 and Co115 using a Tcf-E2 reporter; shows β-catenin is not limiting in SW480 and Co115 colon cancer cell lines.

FIG. 5. E1A inhibits Tcf-dependent transcription. (A) Schematic diagram of the E1A12S mutants. (B-D) Luciferase assays with a wild-type E2 reporter and Tcf-E2 reporters. The “Tcf-E2 mut E3” reporter contains inactivating mutations in the E3 enhancer (9). Cells were transfected with luciferase reporters and plasmids expressing E1A mutants (shown in A). (B) SW480, (C) Co 115, (D) Hct116.

FIG. 6. Luciferase assays in the lung cancer cell line H1299 showing inhibition of Tcf-dependent transcription by mutant forms of E1A. (A) Cotransfection of a Tcf-E1A reporter with various E1A mutants and ΔN-β-catenin. (B) Cotransfection of increasing amounts of p300 plasmid (0.5, 1, or 2 μg) lead to a decrease in Tcf-dependent transcription. (C) Effect of p300, P/CAF and Tip49 on Tcf-dependent transcription in the presence of wild-type and mutant forms of E1A. The values represent the fold activation versus the E1A wild-type reporter in the absence of E1A and ΔN-β-catenin.

FIG. 7. Cytopathic effect assays in different cell lines infected with 10-fold dilutions of wild type Ad5 and Tcf viruses. (A) SW480 cells were infected at a starting multiplicity of 10 pfu/cell and stained 6 days after infection. (B) Co115 and (C) Hct116 were infected at a starting multiplicity of 100 pfu/cell and stained 7 days after infection. (D) HeLa were infected at a starting multiplicity of 100 pfu/cell and stained 8 days after infection.

FIG. 8. Viral burst assays on permissive and non-permissive cell lines. SW480, Hela and SAEC cells were infected with 300 viral particles/cell and lysed 48 hours after infection. The titre of viral particles present in the lysate was measured by plaque assay on SW480. Values were normalised to the wild type Ad5 titre on each cell line. *vCF42 was not tested on SAEC.

FIG. 9. Comparison of sequences of wild type Ad5 E1A promoter and Tcf mutation E1A promoter of the present invention.

FIG. 10. Comparison of sequences of wild type AD5 E4 promoter and Tcf mutation E4 promoter of the present invention.

FIG. 11. Burst Assay results shown as histogram for a number of cell lines infected by Ad5 wt and three viruses of the invention.

FIG. 12. Adenoviruses used in Example 7. The name summarises the structure of the virus: A4=Tcf sites in the E1A and E4 promoters (see also Table 1 herein); C=yCD; I=IRES; S=Ad41 splice acceptor. Size: the size of the viral genome is relative to wild type Ad5. part/pfu: the ratio of particles measured by OD260 to plaque forming units measured on SW480 cells.

FIG. 13. Shows yCD expression was detectable in all three tumor cell lines, with no detectable expression in Normal human lung fibroblasts (HLFs).

FIG. 13a: Western blot for E1A, DBP, fibre and yCD at the indicated times after infection of colon cancer cell lines in presence or absence of ara-C. FIG. 13b: Western blot 48 hours after infection of the same cell lines in absence of ara-C. FIG. 13c: Western blot 48 hours after infection of HLFs in presence or absence of ara-C.

FIG. 14. The exogenous splice acceptor is used correctly in the ASC4 virus.

FIG. 14a: Northern blots of RNA from HT29 cells 48 hours after infection with AIC4 or ASC4 viruses. The blots were probed for yCD or fibre.

FIG. 14b: RT-PCR of the same RNA using primers for the tripartite leader and yCD.

FIG. 14c: Schematic diagram showing the structure of the transcripts in (b). t1-8, transcripts.

FIG. 15. 5-FC increased the toxicity of the yCD viruses in all colon cancer cell lines tested but had only a minor effect in normal cells.

(a) Sensitivity of colon cancer cell lines to 5-FU. Cells were stained four days after addition of the drug. (b to e, g) Cytopathic effect assays using 10-fold dilutions of virus starting from a concentration of 10 pfu/cell. Fresh medium was added four days after infection and cells were stained 5 (SW480), 7 (Hct116 and Hct116^{-/-}), or 8 (HT29 and HLF) days post-infection. (f) 5-FC was added either immediately after infection (early, E) or four days after infection (late, L). Cells were stained 8 days after infection.

FIG. 16. Early treatment with 5-FC is fully compatible with productive infection by yCD viruses.

Viral burst assays in the presence or absence of 5-FC. Colon cancer cell lines were infected with 1 pfu/cell and collected 48 hours post-infection. The titre of viral particles present in the cell pellet and the supernatant were measured by plaque assay on SW480. Values are expressed in pfu produced per pfu used for infection.

### SEQUENCE LISTING

SEQ ID No 1: DNA sequence of Adenovirus type 5.

SEQ ID No 2 to 23: Primers for use in preparing constructs of the invention.

SEQ ID No 24 and 25: cDNAs of toxin producing genes for inclusion in constructs of the invention.

SEQ ID No 26: EMCV internal ribosime entry site sequence for targeting purposes.

Primers

| | |
|---|---|
| GGGTGGAAAGCCAGCCTCGTG | (oCF1) |
| ACCCGCAGGCGTAGAGACAAC | (oCF2) |
| AGATCAAAGGGattaAGATCAAAGG | (oCF3) |
| Gccaccacctcattat | |
| tCCCTTTGATCTccaaCCCTTTGAT | (oCF4) |
| CTagtcctatttatacccggtga | |
| tCCCTTTGATCTccactagtgtgaa | (oCF5) |
| ttgtagttttcttaaaatg | |
| GAACTAGTAGTAAATTTGGG CGTA | (oCF6) |
| ACC | |
| ACGCTAGCAAAACACCTGGGCGAGT | (oCF7) |
| CATTTTCAGTCCC GGTGTCG | (oCF8) |
| ACCGAAGAAATGGCCGCCAG | (oCF9) |
| TCTGTAATGTTGGCGGTGCAGGAAG | (oCF10) |
| ATGGCTAGGAGGTGGAAGAT | (oCF12) |
| and | |
| GTGTCGGAGCGGCTCGGAGG | (oCF13) |
| CAGGTCCTCATATAGCAAAGC | (IR213 E1A antisense) |
| TGTCTGAACCTGAGCCTGAG) | (IR190 E1B sense) |
| CATCTCTACAGCCCATAC | (IR110 E2/E3 sense) |
| AGTTGCTCTGCCTCTCCAC | (IF171 E2/E3 antisense) |
| CGTGATTAAAAAGCACCACC | (IR215 E4 sense) |

Previously disclosed (Wo 00/56909) primers

| | |
|---|---|
| G61 | |
| 5′-TGCATTGGTACCGTCATCTCTA-3′ | Ad 5, 26688 |
| | (E2 region) |
| G62 | |
| 5′-GTTGCTCTGCCTCTCCACTT-3′ | Ad 5, 27882 |
| | (E2 region) |
| G63 | |
| 5′-CAGATCAAAGGGATTAAGATCAAAGGGCC | |
| ATTATGAGCAAG-3′ | |

iPCR, E2 promoter replacement (2×Tcf), upper primer

G64
5′-GATCCCTTTGATCTCCAACCCTTTGATCTAGTCCTTAAGAGTC-3′

iPCR, E2 promoter replacement (2×Tcf), lower primer

G74
5′-GGG CGA GTC TCC ACG TAA ACG-3′

Ad5, 390 (left arm gap repair fragment )

G75
5′-GGG CAC CAG CTC AAT CAG TCA-3′

Ad5, 36581 (right arm gap repair fragment)

G76
5′-CGG AAT TCA AGC TTA ATT AAC ATC ATC AAT AAT ATA
CC-3′

Ad5 ITR plus EcoRI, HindIII and PacI sites

G77
5′-GCG GCT AGC CAC CAT GGA GCG AAG AAA CCC A-3′

Ad 5, 2020 (E1B fragment plus NheI site)

G78
5′-GCC ACC GGT ACA ACA TTC ATT-3′

Ad 5, 2261 (E1B fragment plus AgeI site)

G87
5′-AGCTGGGCTCTCTTGGTACACCAGTGCAGCGGGCCAACTA-3′

iPCR to destroy the E3 NF-1, L1 and L2 binding sites, upper primer

G88
5′-CCCACCACTGTAGTGCTGCCAAGAGACGCCCAGGCCGAAGTT-3′

iPCR to destroy the E3 NF-1, L1 and L2 binding sites, lower primer

G89
5′-CTGCGCCCCGCTATTGGTCATCTGAACTTCGGCCTG-3′

iPCR to destroy the E3 ATF and AP-1 binding sites, upper primer

G90
5′-CTTGCGGGCGGCTTTAGACACAGGGTGCGGTC-3′

iPCR to destroy the E3 ATF and AP-1 binding sites, lower primer

G91
5′-CAGATCAAAGGGCCATTATGAGCAAG-3′

iPCR, E2 promoter replacement (1×Tcf), upper primer

G92
5′-GATCCCTTTGATCTAGTCCTTAAGAGTC-3′

iPCR, E2 promoter replacement (1×Tcf), lower primer

G100
5′-ATGGCACAAACTCCTCAATAA-3′

Ad 5, 27757 (E3 distal promoter region)

G101
5′-CCAAGACTACTCAACCCGAATA-3′

Ad 5, 27245 (E3 distal promoter region)

Mutant leftITR and E1A promoter

catcatcaataatataccttattttggattgaagccaatatgataatgag
gTggtggCCCTTT
GATCTTAATCCCTTTGATCTGGATCCCTTTGATCTCCAACCCTTTGATCT
AGTCCtatttata,

Methods
Adenovirus Mutagenesis

An Ad5 E1A fragment (nucleotides nt 1 to 952) was amplified by PCR from ATCC VR5 adenovirus 5 genomic DNA with primers CGGAATTCAAGCTTAATTAACATCATCAATAATATACC (G76) and GGGTGGAAAGCCAGCCTCGTG (oCF1), cut with PacI, and cloned into the BamHI/PacI sites in pMB1 (see WO 00/56909 incorporated herein by reference) to give pCF4. pMB1 contains the left end of AdS cloned into the EcoRI/SmaI sites of pFL39 ( Bonneaud, N., K. O. Ozier, G. Y. Li, M. Labouesse, S. L. Minvielle, and F. Lacroute. 1991. Yeast. 7:609-15 and Brunori, M., M. Malerba, H. Kashiwazaki, and R. Iggo. 2001. J Virol. 75:2857-65 both incorporated herein by reference.

The endogenous adenoviral sequence from the middle of the ITR to the E1A TATA box was replaced with four Tcf binding sites by inverse PCR with primers tcc

| | |
|---|---|
| AGATCAAAGGGattaAGATCAAAGGGccaccacctcattat | (oCF3) |
| and | |
| tCCCTTTGATCTccaaCCCTTTGATCTagtcctatttatac | (oCF4) |
| ccggtga | |

to give pCF25 (the Tcf sites in the primers are shown in capitals). The final sequence of the mutant ITR and E1A promoter is

catcatcaataatataccttattttggattgaagccaatatgataatg
aggTggtggCCCTTT
GATCTTAATCCCTTTGATCTGGATCCCTTTGATCTCCAACCCTTTGAT
CTAG

TCCtatttata, where the wt Ad5 sequence is in lowercase and the E1A TATA box is underlined. A G to T mutation was introduced just before the first Tcf binding site to mutate the Sp1 binding site ( Leza, M. A., and P. Hearing. 1988J Virol. 62:3003-13 incorporated herein by reference).

The Ad5 E4 fragment (nt 35369 to 35938) was amplified by PCR from VR5 DNA with primers G76 and ACCCGCAGGCGTAGAGACAAC (oCF2), cut with PacI and cloned into the BamHI/PacI sites in pMB1 to give pCF6. To compensate for the mutations introduced in the left ITR, three Tcf binding sites were introduced, and the endogenous sequence (nt 35805 to 35887) was simultaneously deleted by inverse PCR with primers oCF3 and tCCCTTTGATCTccactagtgtgaattgtagttttcttaaaatg (oCF5) to give pCF16 (the Tcf site is shown in capitals and the SpeI site is underlined). The packaging signal was amplified by PCR from pCF6 with primers GAACTAGTAGTAAATTTGGG CGTAACC (oCF6) and ACGCTAGCAAAACACCTGGGCGAGT (oCF7), cut with SpeI/NheI and cloned into the SpeI site in pCF6 to give pCF34. The packaging signal has the same end-to-center orientation as at the left end of the adenoviral genome.

The Δ2-11 mutation was introduced in two steps. First, plasmids pCF4 (wild type E1A promoter) and pCF25 (Tcf-E1A mutant) were cut by SnaBI/SphI following by self ligation to give pRDI-283 and pRDI-284, respectively. Second, the 2-11 region in pRDI-283 and pRDI-284 was deleted by inverse PCR with primers CATTTTCAGTCCC GGTGTCG (oCF8) and ACCGAAGAAATGGCCGCCAG (oCF9) to give pCF61 and pCF56, respectively.

The YAC/BAC vector pMB19 (Gagnebin, J., M. Brunori, M. Otter, L. Juillerat-Jeaneret, P. Monnier, and R. Iggo. 1999 Gene Ther. 6:1742-1750 incorporated herein by reference.) was cut with PacI followed by self ligation to give pCF1, a YAC/BAC vector harbouring a unique PacI site.

In order to produce the gap repair vectors, combinations of left and right adenoviral ends were first assembled and then transferred to the YAC/BAC vector itself. During the first step, pCF34 was cut with EcoRI/SaI and cloned into the Pst/SalI sites of pCF25 to give pRDI-285. Similarly, pCF56 was cut with HindIII/SalI and cloned into the PstI/SalI sites of pCF34 to give pCF46. Finally pCF61 was cut with HindIII/SalI and cloned into the PstI/SalI sites of pCF16 to give pCF52. pRDI-285, pCF46 and pCF52 all contain a cassette with the left and right ends of the genome separated by a unique SalI site. These cassettes were isolated by PacI digestion and cloned into the PacI site of pCF1 to give pCF78, pCF79 and pCF81, respectively. pCF78 had mutant E1A and E4 promoters, pCF79 had mutant E1A and E4 promoters plus the Δ2-11 mutation, and pCF81 has wild-type E1A and E4 promoters plus the Δ2-11 mutation.

vCF11 and vCF22 were constructed by gap repair (Gagnebin, J., M. Brunori, M. Otter, L. Juillerat-Jeaneret, P. Monnier, and R. Iggo. 1999. Gene Ther. 6:1742-1750 incorporated herein by reference.) of pCF78 with VR5 (ATCC) and vMB31 DNA, respectively. vCF42 and vCF62 were constructed by gap repair of pCF79 with VR5 and vMB19 DNA, respectively. vCF81 was constructed by gap repair of pCF81 with vMB31 DNA. The viral DNA was cut with ClaI before gap repair to target the recombination event to a site internal to the mutations at the left end of the genome.

Viral genomic DNA was converted into virus by transfection of PacI digested YAC/BAC DNA into cR1 cells. The viruses were then plaque purified on SW480 cells, expanded on SW480, purified by CsCl banding, buffer exchanged using NAP25 columns into 1 M NaCl, 100 mM Tris-HCl pH 8.0, 10% glycerol and stored frozen at −70° C. The identity of each batch was checked by restriction digestion and automated fluorescent sequencing on a Licor 4200L sequencer in the E1A (nt 1-1050), E1B (nt 1300-2300), E2/E3 (nt 26700-27950) and E4 (nt 35250-35938) regions using primers IR213 (E1A antisense: CAGGTCCTCATATAGCAAAGC), IR190 (E1B sense: TGTCTGAACCTGAGCCTGAG), IR110 (E2/E3 sense: CATCTCTACAGCCCATAC), IF171 (E2/E3 antisense: AGTTGCTCTGCCTCTCCAC) and IR215 (E4 sense: CGTGATTAAAAAGCACCACC). Apart from the desired mutations, no differences were found between the sequence of VR5 and the Tcf viruses. Particle counts were based on the OD₂₆₀ of virus in 0.1% SDS using the formula 1 OD₂₆₀=10¹² particles/ml.

E1A, p300, P/CAF, Tip49 and β-catenin Plasmids

Wild type 12S E1A (pCF9) and E1A mutants ΔpRb (124A,135A), Δp300N (Δ2-11), Δp300C (Δ64-68), Δp400 (Δ26-35), ΔP/CAF (E55), ΔCtBP (LDLA4), and ΔC52 have been described by Alevizopoulos et al (1998) EMBO J. 17:5987-97 and Alevizopoulos et al. (2000) Oncogene. 19:2067-74 and Reid et al. (1998) EMBO J. 17:4469-77 all incorporated herein by reference. All the mutants were provided in a pcDNA3 backbone (Invitrogen, Carlsbad, USA) except the Δp300N and Δp300 C mutants that were isolated with BamHI/EcoRI and cloned into the BamHI/EcoRI sites of pcDNA3. The ΔCR1 mutant (Δ38-68) was made by inverse PCR of pCF9 with primers TCTGTAATGTTGGCGGTGCAGGAAG (OCF10) and ATGGCTAGGAGGTGGAAGAT (oCF12) to give pCF45. The ΔΔ 300-P/CAF double mutant was constructed by three way ligation of BstXI fragments from the single mutants. The ΔN-β-catenin plasmid has been described by Van de Wetering et al. 1997. Cell. 88:789-99 (incorporated herein by reference).

The p300 vector contains HA-tagged p300 expressed from the CMV promoter. The P/CAF expression vector has been described by Blanco et al (1998) Genes Dev. 12:1638-51 The Tip49 and Tip49DN vectors have been described by Wood et al. (2000). Mol Cell. 5:321-30. all incorporated herein by reference.

Cell Lines

ISREC-01 (10), SW480 (ATCC CCL-228) and Co115 (Cottu et al. (1996) Oncogene. 13:2727-30) were supplied by Dr B Sordat. HCT116 (CCL-247), HT29 (HTB-38), 293T were supplied by ATCC. HeLa (CCL-2) were supplied by ICRF. H1299 were supplied by Dr C Prives (Chen et al. (1996). Genes Dev. 10:2438-51.). The cMM1 cell is a H1299 stably transfected tetracycline-responsive minimal CMV promoter (tet-off) line expressing myc-tagged ΔN-β-catenin (Van de Wetering ibid,) pMB92 (the beta-catenin vector) SacII/AccI fragment is cloned into pUHD10-3 SacII/EcoRI. pUHD10-3 is described by Gossen, M. & Bujard, H. (1992). Tight control of gene expression in mammalian cells by tetracycline- responsive promoters. Proc Natl Acad Sci U S A, 89, 5547-51. C7 cells were supplied by Dr J Chamberlain (Amalfitano, A., and J. S. Chamberlain. (1997). Gene Ther. 4:258-63.

To create the cR1 packaging cells, C7 cells were infected with a lentivirus expressing myc-tagged ΔN-β-catenin. Clonetics small airway epithelial cells (SAEC) and SAGM medium were supplied by Cambrex (East Rutherford, USA). All the other cell lines were grown in Dulbecco's Modified Eagle's Medium with 10% fetal calf serum (Invitrogen, Carlsbad, USA).

Luciferase Assays

The E2 reporters were described below. To construct E1A reporters, wild type and mutant E1A promoters were amplified by PCR from pCF4 and pCF25, respectively, with primers G76 and GTGTCGGAGCGGCTCGGAGG (oCF13), cut with HindIII, and cloned into the NcoI/HindIII sites of pGL3-Basic (Promega, Madison, USA). Cells were seeded at 2.5×10⁵ cells per 35-mm well 24 hours before transfection. 4.5 μl of Lipofectamine (Invitrogen, Carlsbad, USA) was mixed for 30 minutes with 100 ng of reporter plasmid, 1 ng of control Renilla luciferase plasmid (Promega, Madison, USA) and 500 ng of vectors expressing E1A, P/CAF, p300 or TIP49. pcDNA3 empty vector was added to equalise the total amount of DNA. In FIG. 5b, 0.5, 1 and 2 μg of p300 vector were used. Cells were harvested 48 hours after transfection and dual luciferase reporter assays performed according to the manufacturer's instructions (Promega, Madison, USA) using a LUMAC Biocounter (MBV). Each value is the mean of one to nine independent experiments done in triplicate and transfection efficiency is normalised to the activity of the Renilla control.

Western Blotting

Cells were infected with 1000 viral particles per cell. Two hours after infection, the medium was replaced. Cells were harvested 24 hours later in SDS-PAGE sample buffer. E1A, ElB55K, DBP and E4orf6 were detected with the M73 (Santa Cruz Biotechnology, Santa Cruz, USA), 2A6 ( Sarnow et al. (1982) Virology. 120:510-7.)), B6 ( Reich et al (1983). Virology. 128:480-4.) and RSA3 (Marton et al (1990) Virol. 64:2345-59) monoclonal antibodies, respectively. Myc-tagged β-catenin was detected with the 9E10 monoclonal antibody (Evan et al (1985) Mol Cell Biol. 5:3610-6) all citations incorporated by reference.

Cytopathic Effect Assay

Cells in six-well plates were infected with ten-fold log dilutions of virus. Two hours after infection, the medium was replaced. After six to eight days (FIG. 6), the cells were fixed with paraformaldehyde and stained with crystal violet.

Virus Replication Assay

Cells in six-well plates were infected with 300 viral particles per cell. Two hours after infection, the medium was replaced. Cells were harvested 48 hours later and lysed by three cycles of freeze-thawing. The supernatant was tested for virus production by counting plaques formed on SW480 cells after 10 days under 1% Bacto agar in DMEM 10% FCS. Each bar in the figures represents the mean +/− SD of triplicate plaque assays.

### EXAMPLE 1

### E1A Promoter Mutations

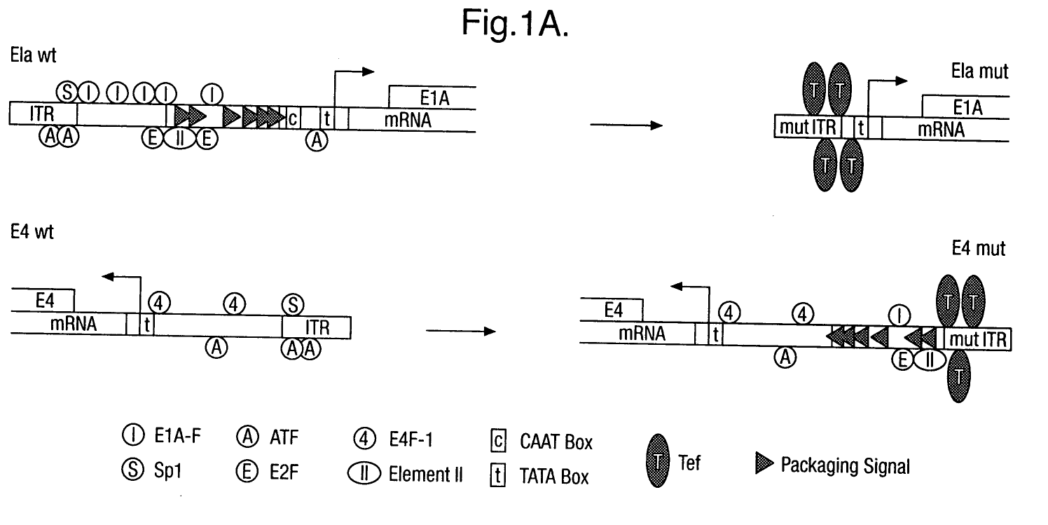

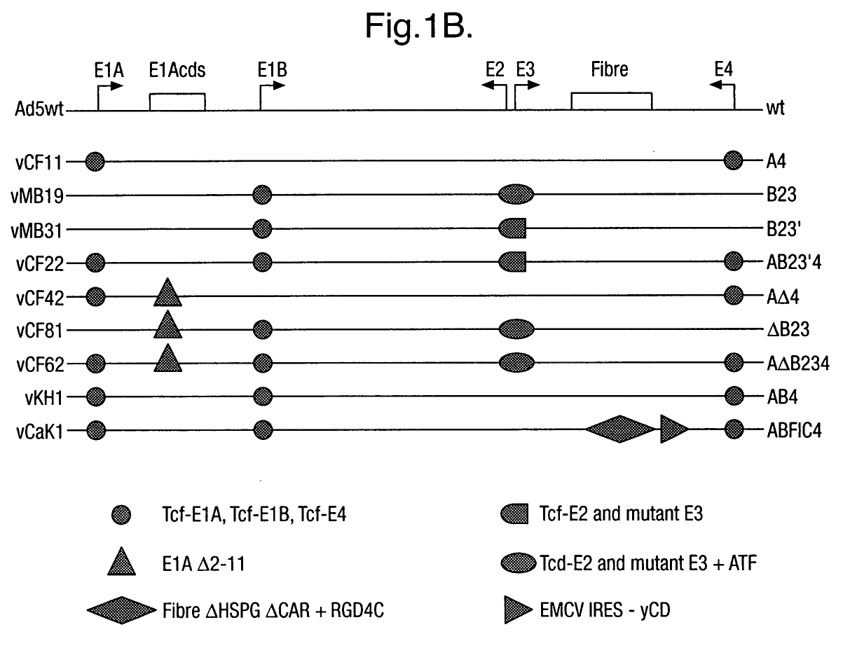

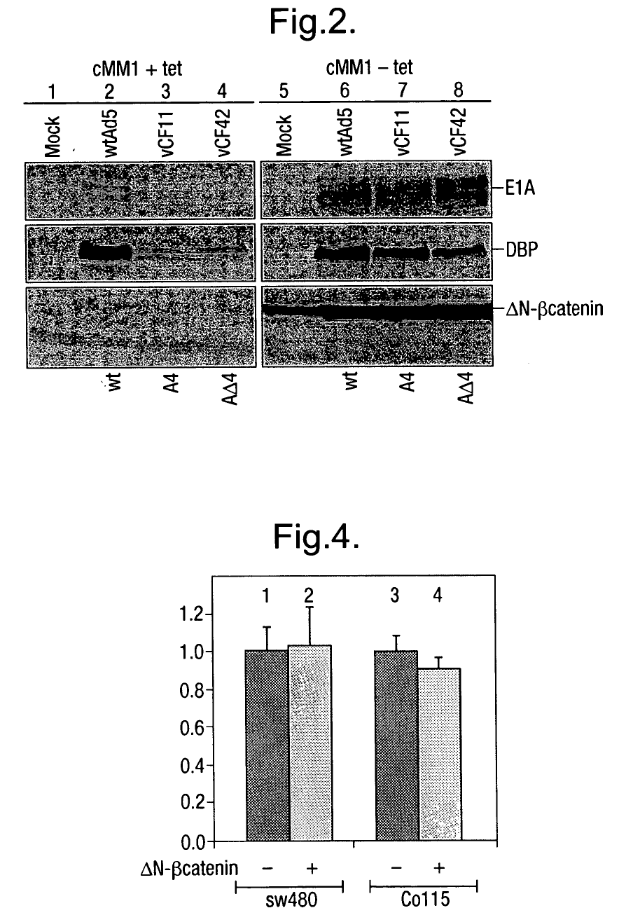

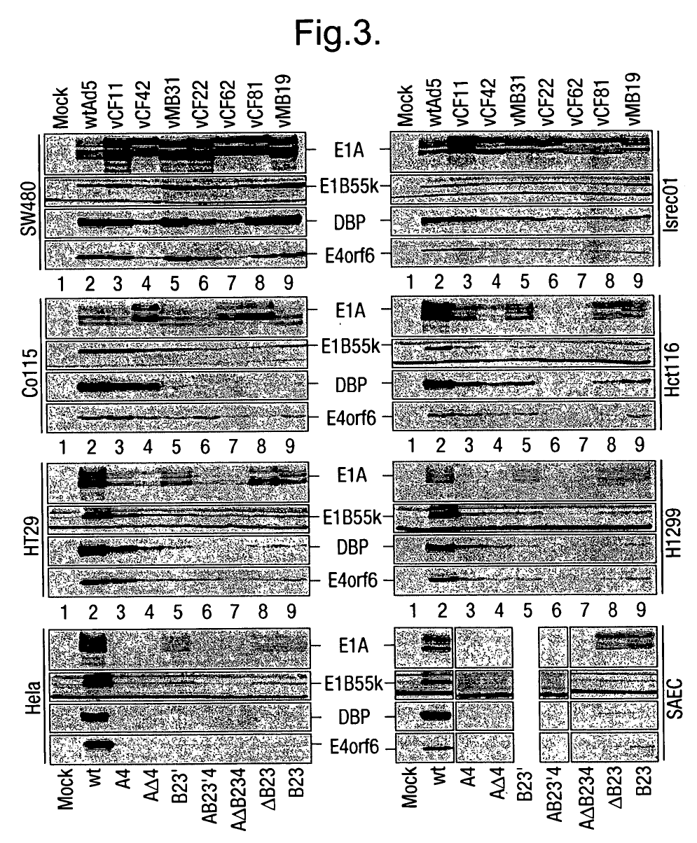

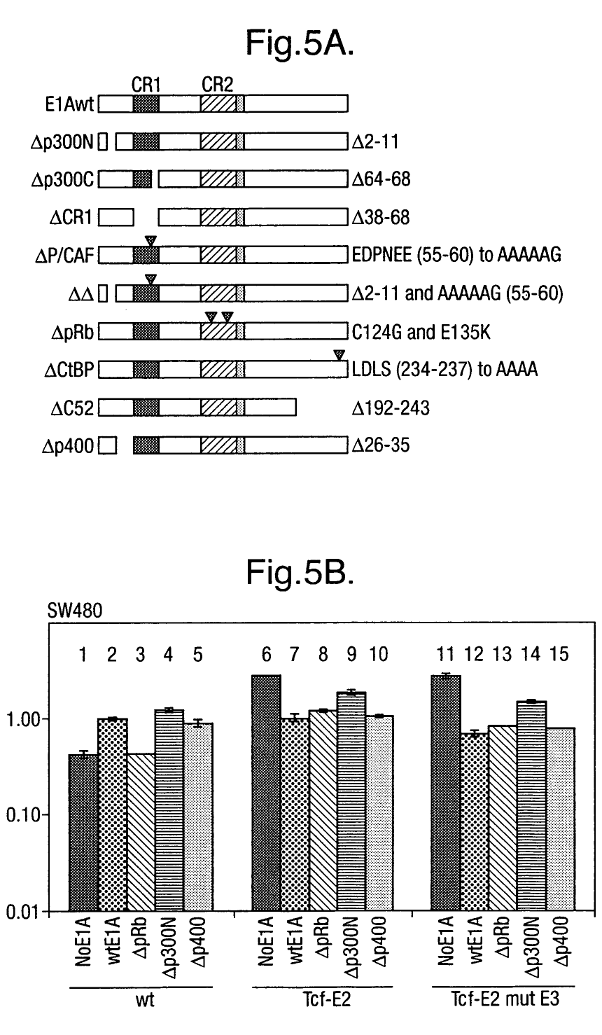

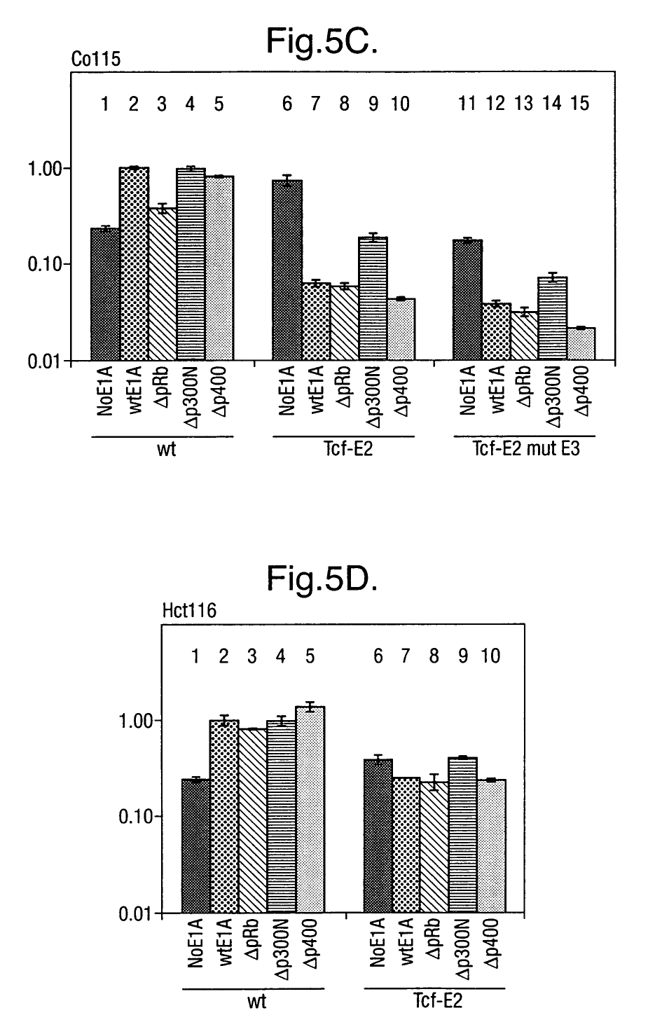

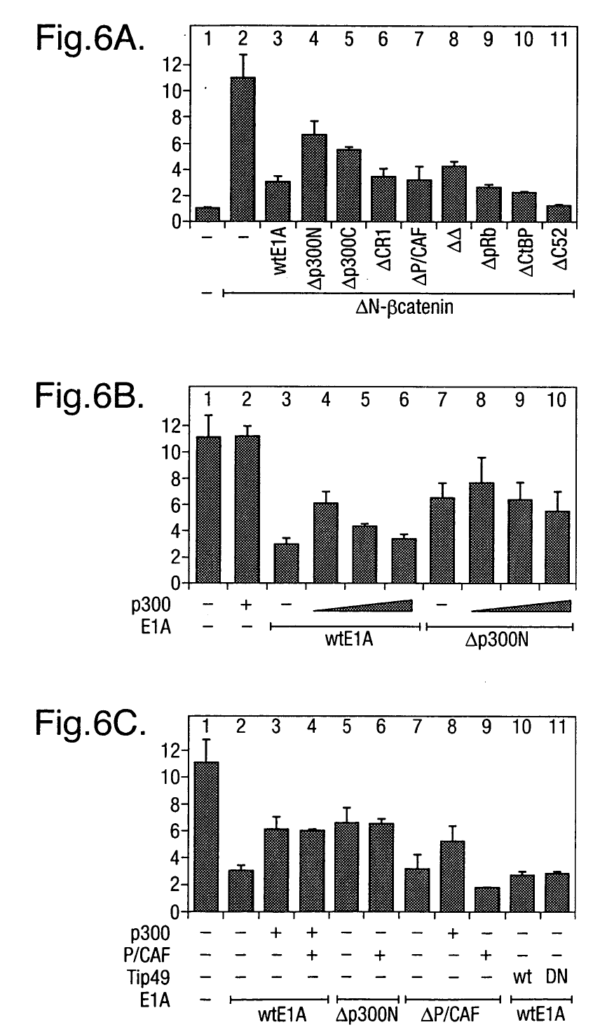

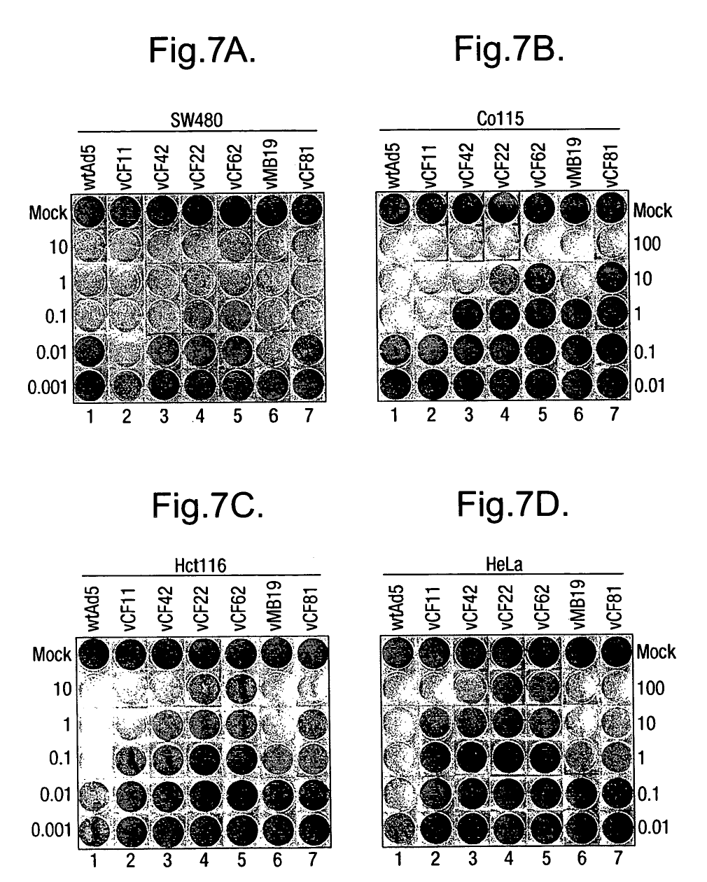

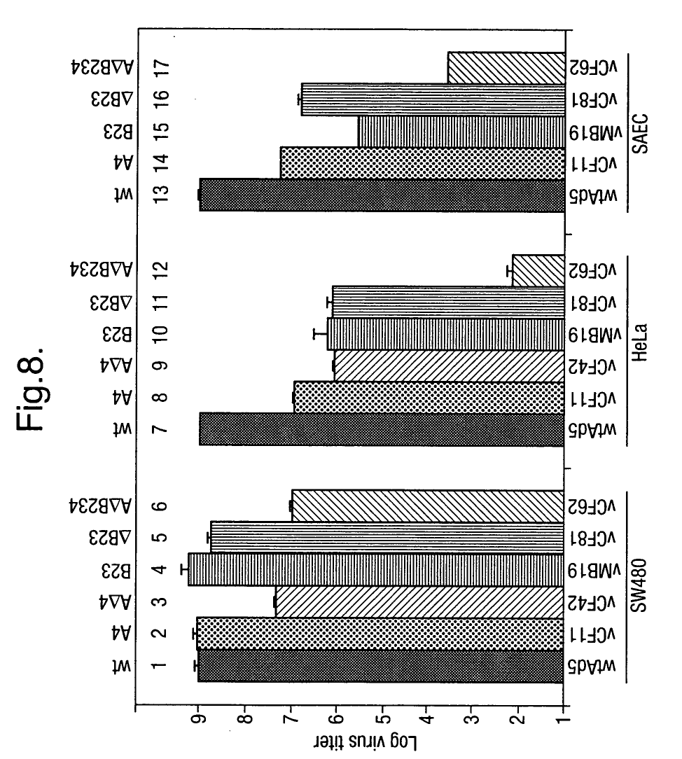

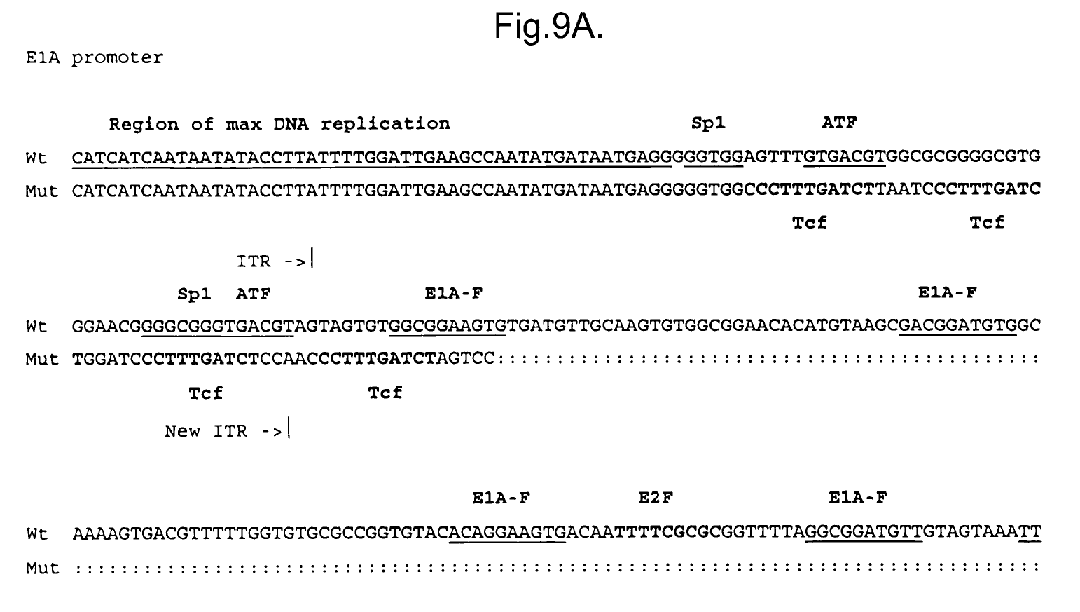

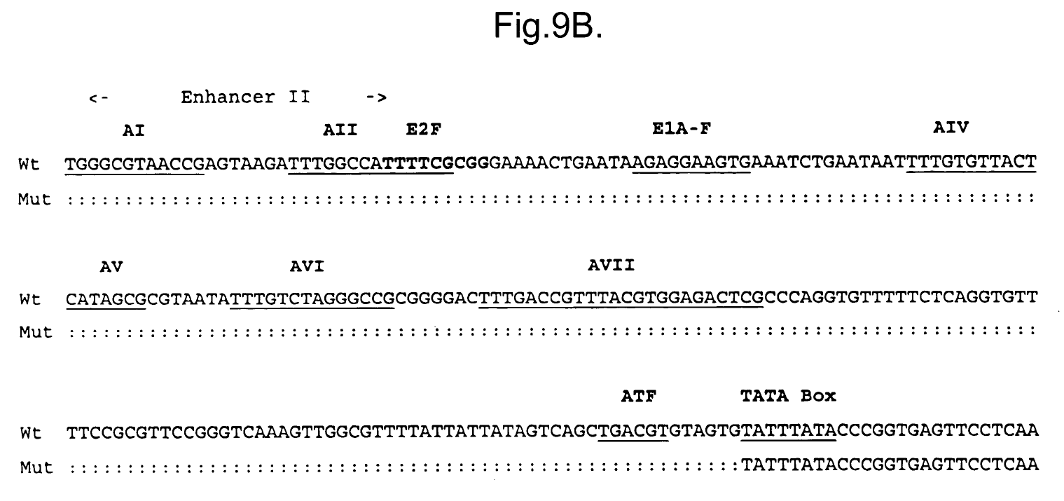

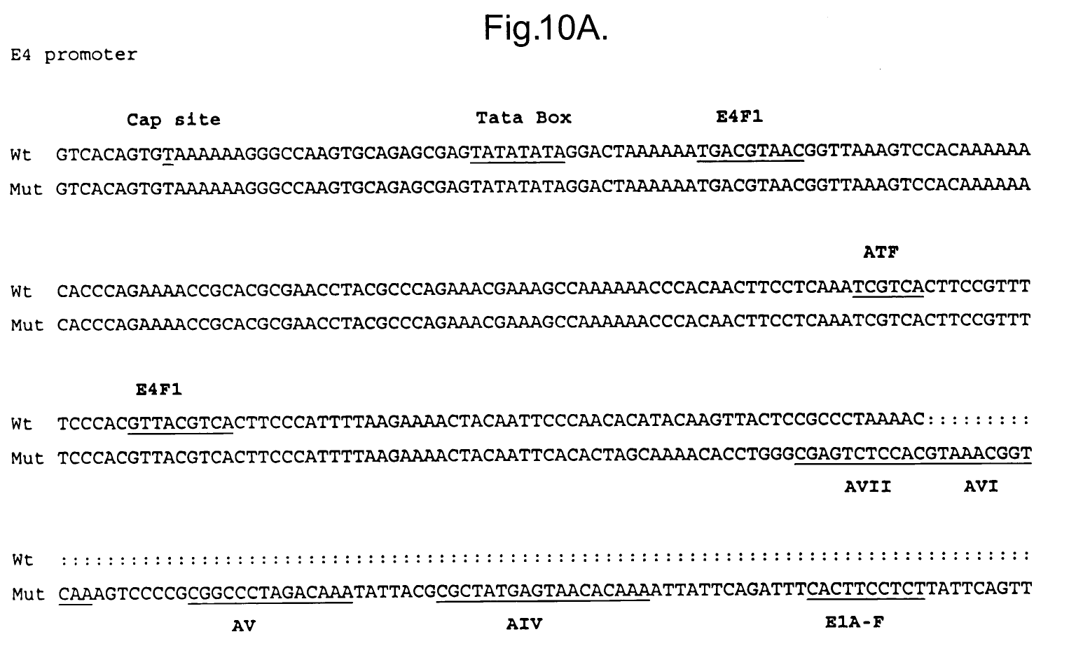

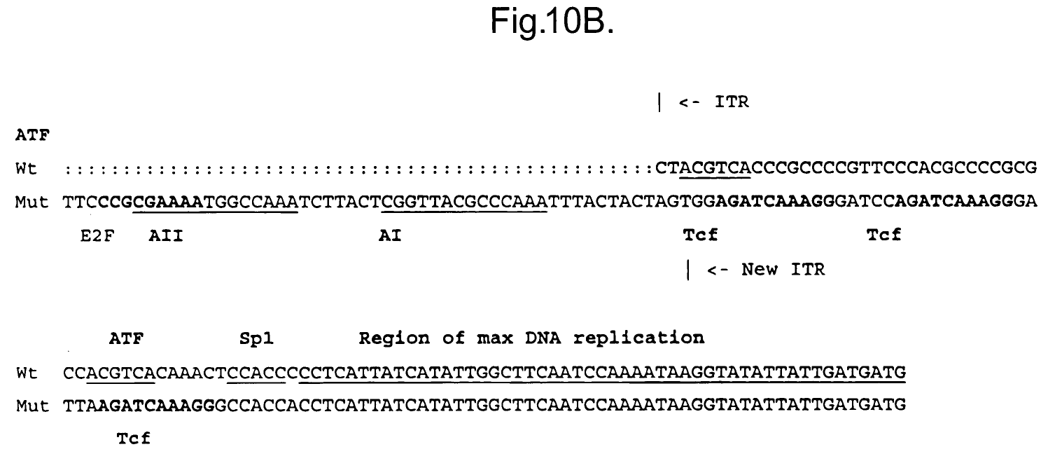

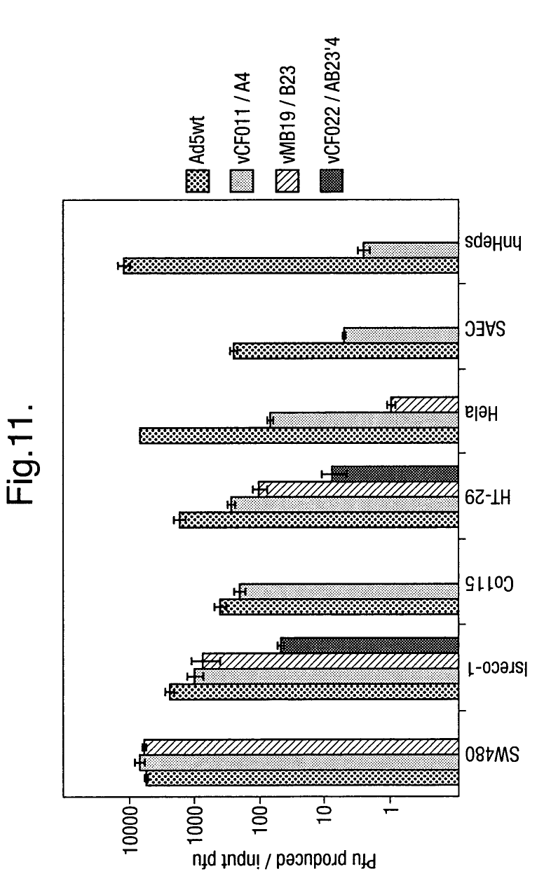

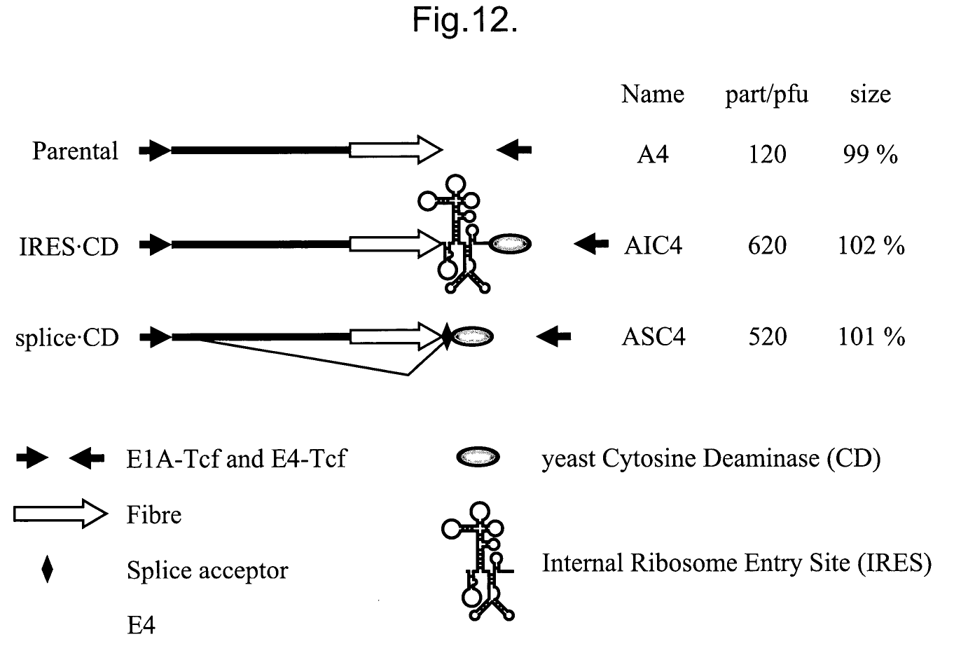

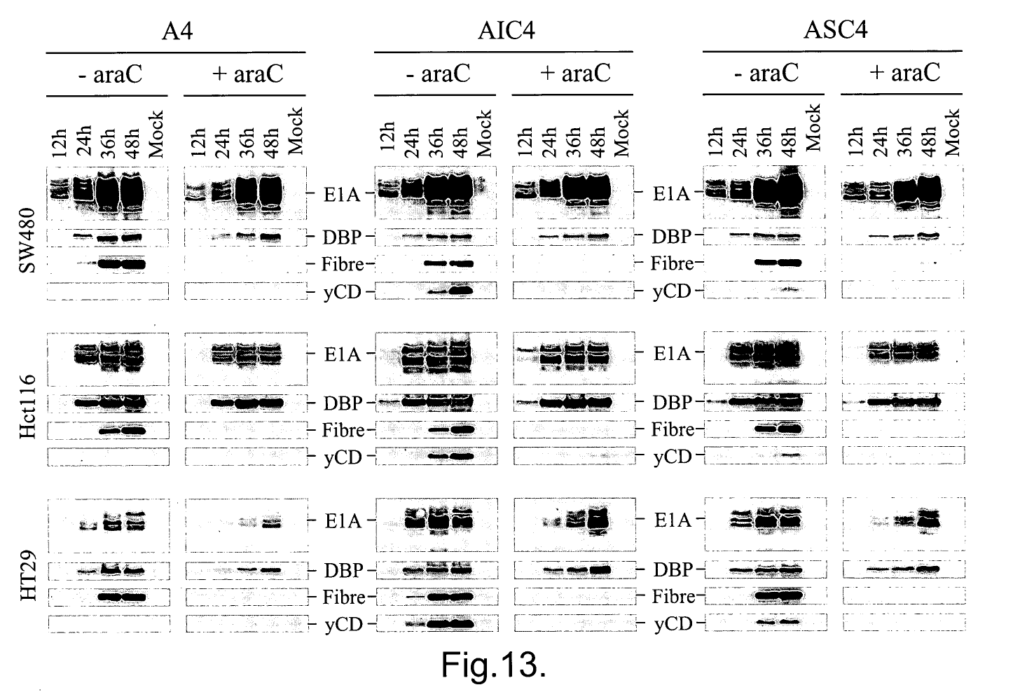

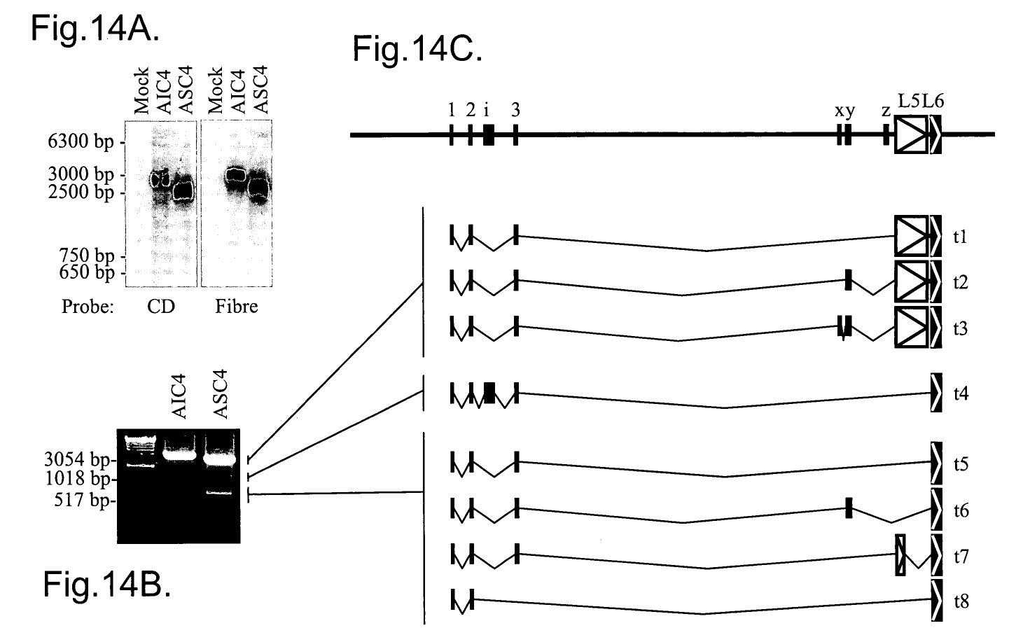

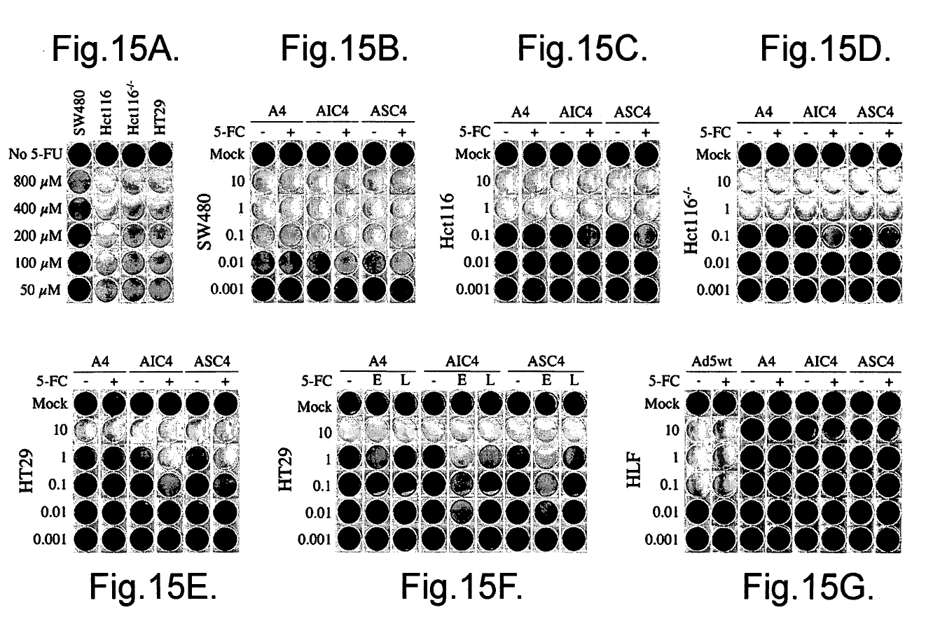

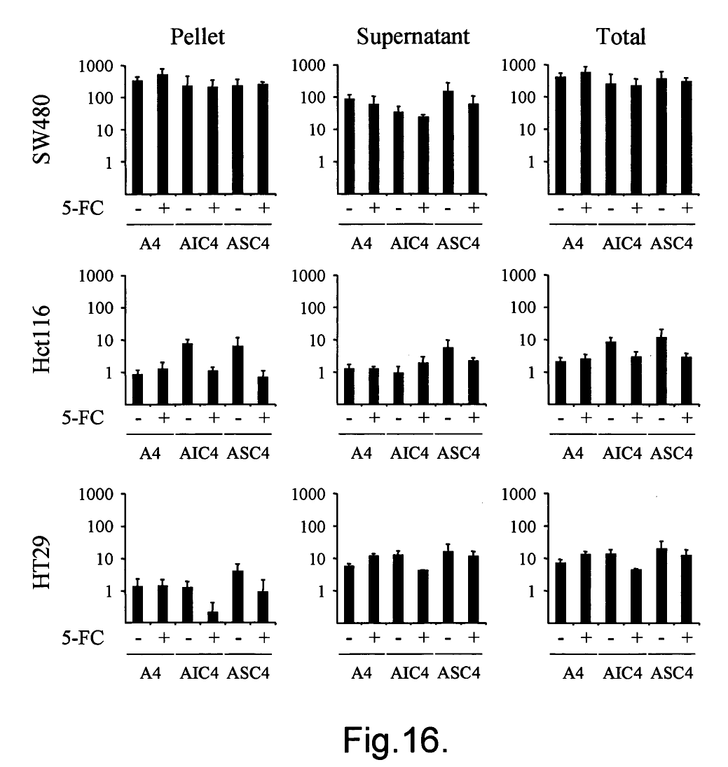

To produce a tightly regulated E1A promoter responding only to wnt signals, the virus packaging signal was transferred to the E4 region and half of the ITR was replaced with Tcf sites. The resulting E1A promoter contains four Tcf sites and a TATA box (FIG. 1). The changes in the ITR do not affect the minimal replication origin (11). Identical changes were made to the right ITR to preserve the ability of the two ITRs to anneal during viral DNA replication. The mutant right ITR contains three Tcf sites followed by the packaging signal and the normal E4 enhancer. Adenoviral genomic DNA was mutagenised in yeast and converted to virus in C7 cells (3) expressing a stable β-catenin mutant. Primary virus stocks were plaque purified and expanded on SW480 cells. The E1A/E4 mutant viruses grew readily on SW480 cells, indicating that the ITR mutagenesis and exchange of the packaging signal are compatible with the production of viable virus. The structure of the viruses used in this study is summarised in table 1.

### EXAMPLE 2

### Tcf-E1A Promoter Viruses

To determine whether the Tcf-E1A promoter responds to activation of the wnt pathway, cMM1 cells were infected with vCF11, the virus with only the E1A/E4 promoter changes. cMM1 cells are a clone of H1299 lung cancer cells expressing ΔN-β-catenin from a tetracycline-regulated promoter. Wnt signalling was activated by removal of tetracycline from the medium (FIG. 2, lanes 5-8, ΔN-β-catenin). This had no effect on E1A expression by wild type Ad5, but induced expression of E1A by vCF11 (FIG. 2, compare lanes 3 & 7, E1A). Since DBP is expressed from the normal E2 promoter in vCF11, the DBP level should rise following activation of wnt signalling, because the normal E2 promoter is activated by E1A. The promoter was weakly active in the absence of E1A in H1299 cells, and showed a moderate increase in activity following induction of ΔN-β-catenin expression (FIG. 2, lanes 3 & 7, DBP). We conclude that the mutant E1A promoter responds to activation of the wnt pathway, and this feeds through to an effect on expression of viral replication proteins.

The effect of the Tcf-E1A/E4 promoter substitutions was then tested on a panel of colon cell lines with active wnt signalling: SW480, ISREC-01 and HT29 have mutant APC; Hct116 has mutant β-catenin; and Co115 has microsatellite instability but the defect in wnt signalling has not been defined (Cottu et al, ibid). Three control cell lines with inactive wnt signalling were tested: H1299, HeLa and low passage human small airway epithelial cells (SAEC). E1A was detectable by western blotting 24 hours after vCF11 infection of all of the colon cell lines but not the H1299, HeLa or SAEC (FIG. 3, lane 3, E1A). Relative to wild type Ad5, the level of E1A expression was higher in SW480 and ISREC-01, the same in Co115 and lower in HT29 and Hct116 (FIG. 3, compare lanes 2 & 3, E1A). The hierarchy of responsiveness of the Tcf-E1A promoter in the different cell lines was thus the same as with the Tcf-E2 viruses of WO 00/56909 but the level of expression relative to the normal promoter was higher for E1A than E2. Since the E1B and E2 enhancers are wild type in vCF11, these transcription units should be inducible by E1A. The E4 promoter in vCF11 is potentially able to respond to both E1A and Tcf. To test this, the blots were probed for E1B 55k, DBP and E4 orf6. Consistent with the E1A results, all three proteins were expressed normally in SW480, ISREC-01 and Co115, and undetectable in HeLa and SAEC (FIG. 3, compare lanes 2 & 3). Despite the absence of E1A expression, all three proteins were expressed weakly in H1299 cells, suggesting that these cells contain an endogenous activity which can substitute for E1A. Compared to wild type infections, the level of E1B 55k, DBP and E4 orf6 was slightly reduced in HT29 and more substantially reduced in Hct116 cells infected with vCF11 (FIG. 3, compare lanes 2 & 3).

### EXAMPLE 3

### Viruses with Tcf Sites in Multiple Early Promoters

To test the effect of regulating E1A expression in the context of the previous generation of Tcf viruses, cells were infected with vMB31 (Tcf-E1B/E2) and vCF22 (Tcf-E1A/E1B/E2/E4; FIG. 3, compare lanes 5 & 6). E1A and E4 orf6 expression were well preserved in SW480, ISREC-01 and Co115 infected with vCF22, but DBP expression was maintained only in SW480 and ISREC-01, and even there it was slightly lower with vCF22 than wild type Ad5 (FIG. 3, compare lanes 2 and 6, DBP). In the remaining cell lines, DBP expression was undetectable with vCF22. Insertion of Tcf sites in the E1A, E1B, E2 and E4 promoters in vCF22 abolished the E1A-independent expression of E1B 55K, DBP and E4 orf6 seen in H1299 infected with vCF11 (FIG. 3, compare lanes 3 and 6, H1299). We conclude that insertion of Tcf sites into multiple early promoters produces an extremely selective virus but one with reduced activity even in some colon cell lines.

### EXAMPLE 4

### Inhibition of Tcf-Dependent Transcription by E1A

The defect in early gene expression from the Tcf viruses in the semi-permissive cell lines is not restricted to a single promoter. Instead, there appears to be a general defect in activation of viral Tcf promoters. This can be partly explained by generally weaker Tcf activity. The reason for this is unclear, but it does not reflect a lack of wnt pathway activation per se, since the semi-permissive cell lines all contain mutations in either APC or β-catenin, and the Tcf-E2 transcriptional activity measured by luciferase assay is not increased by transfection of exogenous ΔN-β-catenin (FIG. 4a).

An alternative explanation for the semi-permissivity of some cell lines is that E1A could be inhibiting the viral Tcf promoters, for example by inhibiting p300, which is a coactivator of Tcf-dependent transcription (Leza and Hearing. (1988). J Virol. 62:3003-13, Takemaru (2000) J Cell Biol. 149:249-54).

To determine whether E1A inhibits the viral Tcf promoters, we performed transcription assays using the Tcf-E1A and Tcf-E2 promoters coupled to the luciferase gene. In SW480, the Tcf-E2 promoter was more active than the wild type E2 promoter in the absence of E1A (FIG. 4b, lanes 1 & 6), and gave almost exactly wild type activity in the presence of E1A (FIG. 4b, lanes 2 & 7). This convergence was due to increased wild type E2 promoter activity and decreased Tcf-E2 promoter activity in the presence of E1A. Mutation of the E3 promoter is required to produce a tightly regulated Tcf-E2 promoter, because the E3 promoter is adjacent to the E2 promoter (9). E3 mutation reduced the activity of the E2 promoter slightly in SW480 cells transfected with E1A, but the activity was still close to that seen with the wild type promoter (FIG. 4b, lanes 2 & 12). The high activity of the Tcf-E2 promoter in SW480 probably explains why this cell line is permissive for all of the Tcf viruses. In contrast, the level of Tcf-E2 activity in the presence of E1A was substantially below the wild type level in Co115 and Hct116 cells (FIGS. 4c & d, lanes 2, 7 & 12).

To determine the mechanism of inhibition, we tested different E1A mutants. Mutation of the Rb binding site in E1A impaired transactivation of the wild type E2 promoter in SW480 and Co115 (FIGS. 4b & c, lane 3) but not Hct116 cells (FIG. 4d, lane 3), whereas mutation of the p300 or p400 binding sites had little effect on transactivation of the wild type promoter by E1A in all three cell lines (FIGS. 4b, c & d, lanes 4 & 5). Reduced transactivation by an E1A mutant unable to bind Rb is expected, given the presence of E2F sites in the E2 promoter. The Tcf sites replace the normal enhancer in the Tcf-E2 promoter. In all three cell lines the Rb and p400 binding site mutations did not relieve inhibition of the Tcf promoters by E1A (FIG. 4b, c & d, lanes 8, 10, 13 & 15). The only mutation to have an effect was the p300 binding site mutation (E1A Δ2-11, labelled Δp300N), and in SW480 and C115 the maximum recovery never exceeded 50% of the lost activity (FIGS. 4b, c & d, lanes 9 & 14). Mutation of E1A amino acid 2 to glycine (R2G), which also blocks p300 binding, had the same effect (data not shown).

### EXAMPLE 5

### Analysis of Additional E1A Mutants

To explore possible explanations for the incomplete recovery of activity after mutation of the p300 binding site in E1A, additional luciferase assays were performed in H1299 cells (FIG. 5). The Tcf-E2 promoter was activated 10-fold by ΔN-β-catenin (FIG. 5a, compare lanes 1 & 2), and this was inhibited by E1A (FIG. 5a, lane 3). p300 binds to two sites in E1A and mutation of either site partially relieved the inhibition of Tcf-dependent transcription (E1AΔp300N and Δp300C, FIG. 5a, lanes 4 & 5). The C-terminal p300 binding site lies within conserved domain 1 (CR1), but deletion of the entire domain did not restore activity (FIG. 5a, lane 6). This suggests that there may be a positively acting factor which binds somewhere in CR1. To determine whether the E1A Δp300N mutation only partially restored activity because it did not completely block p300 binding, we cotransfected increasing amounts of p300 with E1A (FIG. 5b). Exogenous p300 reversed the inhibition of promoter activity to the same extent as mutation of the p300 binding site (FIG. 5b, lanes 4 & 7), and the effects of the Δp300N mutation and p300 transfection were not additive (FIG. 5b, lane 8). Large amounts of exogenous p300 reduced promoter activity (FIG. 5b, lanes 5, 6, 9 & 10), suggesting that a cofactor was being titrated. P/CAF is a candidate for this cofactor because it is a histone acetyltransferase (HAT) that binds to p300, and the coactivation of Tcf by p300 does not require intrinsic p300 HAT activity. Since E1A inhibits P/CAF we tested whether mutation of the P/CAF binding domain in E1A relieved inhibition of Tcf activity by E1A, but saw no effect (FIG. 5a, lane 7). P/CAF was not limiting because cotransfection of P/CAF and wild type or ΔP/CAF mutant E1A also failed to restore activity (FIG. 5c, lanes 4 & 9). To test whether p300 and P/CAF act together, an E1A gene with mutations in the binding sites for both HATs was constructed (labelled ΔΔ in FIG. 5), but this mutant also failed to relieve the repressive effect of E1A (FIG. 5a, lane 8), as did cotransfection of P/CAF and E1A mutant in the p300 binding site (FIG. 5c, lane 6) or cotransfection of p300 and E1A mutant in the P/CAF binding site (FIG. 5c, lane 8).

As in colon cells (FIG. 4), mutation of the Rb binding site in E1A had no effect on repression of Tcf-dependent transcription (FIG. 5a, lane 9). CtBP and TIP49 have both been implicated in transcription activation by Tcf (Bauer et al. (2000). EMBO Journal. 19:6121-6130; Brannon et al (1999). Development. 126:3159-70), but neither mutations in E1A which abolish CtBP binding (ACtBP, AC52; FIG. 5a, lanes 10 & 11) nor transfection of wild type or dominant negative TIP49 (FIG. 5c, lanes 10 & 11) could overcome the repressive effect of E1A. In conclusion, the E1A mapping studies showed that mutation of the p300 binding domain could restore about half of the Tcf activity lost upon E1A expression, but the remaining repressive effect could not be mapped to a known domain in E1A.

### EXAMPLE 6

### E1AΔp300N Mutant Tcf Viruses

To test whether deletion of the p300 binding site in E1A would increase the activity of the Tcf promoters in the context of the virus, the Δp300 N mutation was introduced into the Tcf-E1A, Tcf-E1B, Tcf-E2 and Tcf-E4 viruses (table 1). For the Tcf-E1A promoter, inhibition of p300 by E1A should inhibit expression of E1A itself. This was tested by infecting the cMM1 cell line with vCF11 and vCF42, the Δp300N derivative of vCF11, in the presence and absence of tetracycline. Consistent with there being negative feedback by E1A on its own expression, the level of E1A after activation of wnt signalling was higher with vCF42 than vCF11 (FIG. 2, compare lanes 7 & 8, E1A). Despite the increase in E1A expression, there was no difference in DBP expression, possibly because the Δp300N mutant is defective in some other function required for activation of the wild type E2 promoter (FIG. 2, compare lanes 8 & 9, DBP). The multiply mutated viruses were then tested on a panel of cell lines (FIG. 3). The effect of the Δp300N mutation can best be appreciated by comparing matched pairs of viruses: vCF11 vs vCF42 (FIG. 3, lanes 3 & 4); vMB19 vs vCF81 (FIG. 3, lanes 9 & 8); and vCF22 vs vCF62 (FIG. 3, lanes 6 & 7). In each case the latter is derived from the former by deletion of the p300 binding site in E1A (the only exception is that the E3 promoter ATF site in present in vCF22 but absent in vCF62). In almost every case the Δp300N mutation actually reduced the level of expression of E1B 55K, DBP and E4 orf6. The only promoter whose activity was reasonably well maintained was the Tcf-E1A promoter (FIG. 3, lanes 4 & 7, E1A). The wild type E1A promoter was also little affected by the E1AΔp300N mutation (FIG. 3, lane 8, E1A). The most comprehensively mutated virus (vCF62, FIG. 3, lane 7) was completely inactive in the control cell lines (H1299, HeLa and SAEC), but also severely attenuated in the semi-permissive colon lines (Co115, HT29 and Hct 116). The E1AΔp300N mutation did not increase E1B 55K or DBP expression in any of the viruses with Tcf-E1B and Tcf-E2 promoters (FIG. 3, compare lanes 6 vs 7, and 9 vs 8). We conclude that in the context of the virus the E1AΔp300N mutation does not rescue the defect in Tcf promoter activity in the semi-permissive cell lines.

Since this result was unexpected, we also tested the new viruses in cytopathic effect and burst assays. In the most permissive colon cell line, SW480, both vCF11 and vMB19 were at least 10-fold more active than wild type Ad5 in burst assays (FIG. 6a, compare lane 1 with lanes 2 & 6). For the less engineered viruses the p300 mutant was about 10-fold less active than the corresponding virus expressing wild type E1A (FIG. 6a, compare lanes 2 vs 3, and 6 vs 7).

Only for the virus with Tcf sites in the E1A, E1B, E2 and E4 promoters was the p300 mutant virus as active as the parent (FIG. 6a, compare lanes 4 vs 5), but these viruses were 100-fold less active than the virus with only the Tcf-E1A/E4 changes (vCF11, FIG. 6a, lane 2). vCF11 showed wild type activity on Co115 (FIG. 6b, compare lanes 1 vs 2). This is 10-fold better than the previous best virus, vMB19 (FIG. 6b, lane 7). In Hct116, the situation was reversed: vMB19 was slightly better than vCF11, but wild type was better than either Tcf virus (FIG. 6c, lanes, 1, 2 & 7). In Co115, all of the p300 mutant viruses were 10-fold less active than the corresponding viruses with wild type E1A (FIG. 6b, compare lanes 2 vs 3, 4 vs 5, and 6 vs 7). All of the Tcf viruses were substantially less active than wild type Ad5 on HeLa cells, which lack Tcf activity (FIG. 6d). The most engineered viruses failed to produce foci on HeLa even after infection with 100 pfu/cell (FIG. 6d, lanes 4 & 5). The effect of mutation of the p300 binding site in E1A was less obvious than on permissive cells. Overall, the best virus was vCF11, which was 10-fold less active than vMB19 and 1000-fold less active than wild type Ad5 on Hela cells (FIG. 6d, lanes 1, 2 & 6). Since vCF11 is 10-fold more active than wild type Ad5 on SW480, its overall selectivity for the most permissive colon cells is 10,000-fold relative to wild type Ad5.

In burst assays, the effect of the p300 binding site mutation was specific to the virus and the cell line. In SW480, the mutation reduced burst size 50-fold in the Tcf-E1A/E4 backbone (FIG. 7, compare lanes 2 & 3), but had no effect in the Tcf-E1B/E2 backbone (FIG. 7, compare lanes 4 & 5). This difference may be due to the fact that E2 promoter requires E1A function in vCF42, where the wild type E2 enhancer is activated by ATF and E2, but not in vCF81, where the E2 enhancer is replaced by Tcf sites. The virus with Tcf sites in all the early promoters and the Δp300 mutation in E1A (vCF62) was 100-fold less active than wild type in SW480, which was only slightly worse than vCF42 (FIG. 7, compare lanes 3 & 6). There was a striking reduction in vCF62 burst size in the non-permissive cells (10⁷-fold in HeLa cells, 10⁵-fold in SAEC; FIG. 7, lanes 12 & 18). The remaining Tcf viruses showed 100 to 5000-fold reduced burst size in HeLa and SAEC. The Δp300 mutation again reduced burst size in the virus with E2 driven by E1A (FIG. 7, compare lanes 8 & 9), but actually increased burst size (albeit from a very low level) in SAEC when the E2 promoter was driven by Tcf (FIG. 7, compare lanes 16 & 17).

Comparative Viruses of WO 00/56909

The inventors have previously constructed as follows as refered to in WO 00/56909, incorporated herein by reference. Viruses with the amino-terminus of E1B 55K fused to GFP (comparative virus LGM), with replacement of the E2 promoter by three Tcf sites (virus Ad-Tcf3), and with the two combined (virus LGC). The inventors have also constructed viruses with replacement of the E2 promoter by four Tcf sites alone (virus vMB12), with replacement of the E2 promoter by four Tcf sites combined with silent mutations in the E3 promoter, particularly to NF1, NFκB, AP1, and ATF sites (virus vMB14), and with replacement of the E2 promoter by four Tcf sites combined with silent mutations in the E3 promoter, particularly to NF1, NFκB, AP1, but not ATF sites (virus vMB13). The inventors have also constructed viruses with replacement of the Sp1 site in the E1B promoter with four Tcf sites in a wild type adenovirus backbone (virus vMB23), in a vMB12 backbone (virus vMB27), in a vMB13 backbone (virus vMB31) and in a vMB14 backbone (virus vMBl19).

### EXAMPLE 7

### vCF11 Viruses (A4 Backbone) with yCD in the Major Late Show 10 Fold Increase in Toxicity

We have shown that it is possible, if desired, to enhance the toxicity of viruses of the invention by inserting a toxin or prodrug activating gene (so called “suicide genes”), such as yeast cytosine deaminase into the major late transcript. In this example the yCD was inserted after the fibre gene in the major late transcript, see the late region of construct vCaK1 on FIG. 1B and Table 1. yCD was expressed using either an internal ribosome entry site (IRES) or by alternative splicing, see FIG. 12. Both approaches resulted in yCD expression restricted to the period after viral DNA replication. The IRES virus gives higher yCD expression on western blots. Cytopathic effect assays show that both viruses have 10-fold increased toxicity in the presence of the prodrug 5-fluorocytosine (5-FC), which is converted to 5-fluorouracil (5-FU) by yCD. Viral burst size was only slightly impaired by 5-FC and toxicity was higher following early treatment. The largest gain in toxicity was seen in HT29 cells, which are the least permissive colon cancer cells for the parental virus, indicating that the new 5-FC/yCD viruses may have broad applications for colon cancer therapy.

Virus Constructs

Yeast cytosine deaminase was used because it has a lower Km and higher Vmax than the bacterial enzyme. The yCD coding sequence was inserted at the end of the L5 transcript in A4, which has Tcf sites in the E1A and E4 promoters. Two viruses were produced (“AIC4” and “ASC4”, FIG. 12). The AIC4 virus uses the encephalomyocarditis virus (EMCV) IRES to convert the L5 transcript into a bicistronic mRNA. The ASC4 virus uses the splice acceptor sequence from the Ad41 short fibre gene to splice the yCD cassette onto the tripartite leader exons of the major late transcript. The yCD insertion contributes 520 bp to ASC4 and 1071 bp to AIC4, for a total genome size that is only slightly larger than normal (the A4 backbone is smaller than Ad5). Both viruses grow well on SW480 cells, which have high Tcf activity and were used as producer cells. The viruses have a particle/pfu ratio approximately 5-fold higher than the parental virus, an increase that could be explained by the increase in genome size or a slight delay in fibre expression (see below).

yCD is Expressed with Late Kinetics

SW480 is a colon cancer cell line in which the A4 virus replicates slightly better than wild-type Ad5; Hct116 and HT29 are colon cancer cell lines with lower Tcf activity which are less permissive for A4 replication. To check yCD expression from the Tcf viruses, cell extracts were collected at various times after infection and western blots were probed for yCD and viral proteins. yCD expression was detectable in all three cell lines, with stronger expression from AIC4 than ASC4 (FIG. 13a). Direct comparison of the three viruses on the same blot 48 hours after infection confirmed the impression that AIC4 gives higher yCD expression than ASC4 (FIG. 13b). All of the viruses gave similar fibre and DBP expression at this time; the level of E1A was higher with SW480 than the other cell lines, in keeping with the higher Tcf activity in this line. To determine whether yCD is expressed as a late gene, cells were treated with cytosine arabinoside (ara-C) to inhibit viral replication. This had no effect on expression of early genes (E1A and DBP) but blocked expression of yCD and fibre, showing that these behave as late genes.

Normal human lung fibroblasts (HLFs) were infected with the yCD viruses to test whether the A4 backbone retains its specificity for tumor cells after insertion of the transgene. There was no detectable yCD expression, but both yCD viruses expressed DBP, and the ASC4 virus even expressed a small amount of fibre (FIG. 13c). This could be caused by contamination of the prep with wild type virus. This was excluded by rigorous checking of the virus preps using PCR primers specific for the wild type E1A promoter. DBP expression was not blocked by ara-C, showing that DBP was expressed from an early promoter. It is possible that the yCD sequence contains enhancer elements which can act either at a distance on the E2 early promoter or locally on an uncharacterised early promoter near the site of transgene insertion. The transgene may also include splice sites that would allow splicing from E4 onto E2.

The DBP expression in HLFs probably does lead to some virus replication and yCD expression, which can be seen as a slight decrease in the confluence of 5-FC-treated HLFs infected with the highest dose of virus. Despite this reduction in selectivity, there remains a greater than 100-fold difference between all the Tcf viruses and wild type Ad5. It is worth pointing out in this context that the Tcf virus backbone used for these experiments has the least selectivity of our family of Tcf viruses. If greater selectivity is required we have ample scope to increase it by adding Tcf sites to other early promoters.

The Exogenous Splice Acceptor is used Correctly in the ASC4 Virus

To determine whether the yCD cassette functions correctly as an L6 transcript in the ASC4 virus, the structure of the yCD mRNA was examined by northern blotting and RT-PCR. RNA was extracted from infected HT29 cells and hybridised with fibre and yCD probes (FIG. 14a). AIC4-infected cells gave a 3 kb band with both probes that had the expected size of the fibre-IRES-yCD mRNA. The structure of the mRNA was confirmed by sequencing. The ASC4-infected cells gave a 2.5 kb band with both probes that was bigger than the expected wild type fibre or yCD mRNAs (2.0 kb and 0.7 kb, respectively). To ascertain the nature of this RNA, an RT-PCR was performed with primers in the tripartite leader and yCD (FIG. 14b). This confirmed the presence of the major 3.0 kb and 2.5 kb transcripts observed on the northern blot. The RT-PCR from the ASC4-infected cells also showed smaller bands potentially corresponding to correctly spliced L6 RNA. The 2.5, 1.0 and 0.7 kb PCR products from the ASC4-infected cells were cloned and sequenced.

A schematic description of the observed transcripts is shown in FIG. 14c. The 2.5 kb band corresponds to yCD transcripts that contain the fibre gene preceded by the tripartite leader either alone (labelled t1 in FIG. 14c) or combined with the x and y leaders (t2, t3). The presence of these transcripts is explained by failure of the prototypic L5 transcripts to use the polyA signal placed between the fibre and yCD genes. The lower bands correspond to mRNAs that use the exogenous Ad41 splice acceptor to create the desired new L6 transcript. The tripartite leader was correctly used, either alone (t5) or in conjunction with other leaders (t4 and t6). Two additional minor transcripts were observed (t7 and t8), which nevertheless still used the Ad41 acceptor. We conclude that the Ad41 splice acceptor is functioning correctly but weakly and that the polyA signal between fibre and yCD is used inefficiently if at all. The small amount of correctly spliced yCD transcripts readily explains the lower yCD expression seen on western blots with the ASC4 than the AIC4 virus (FIGS. 13a & b).

The Cytotoxicity of the vCD Viruses is Increased by 5-Fluorocytosine

Before testing the toxicity of the yCD viruses, we first looked at the sensitivity of different colon cancer cell lines to 5-fluorouracil (FIG. 15a). Cells were grown in the presence of various 5-FU concentrations for 4 days and stained with crystal violet, to mimic the readout of a CPE assay. SW480 cells were at least 10-fold more resistant to 5-FU than the other cell lines. Hct116 cells with a homozygous deletion of the tumor suppressor gene p53 show a greatly reduced apoptotic response to 5-FU but were only slightly more resistant than the parental cells in this assay. The cells were then infected with 10-fold dilutions of virus in the presence or absence of the prodrug 5-FC (FIG. 15b to 15e). In every cell line tested, 5-FC had no effect on the toxicity of the parental A4 virus but increased the toxicity of both yCD viruses ˜10-fold (compare lanes + and −). The biggest effect was seen in HT29, which express yCD the best but replicate the virus the worst. There was no correlation between p53 status or the initial sensitivity of the cell lines to 5-FU and the response to combination therapy. This suggests that the two treatments act synergistically. The gain in cytotoxicity was comparable with AIC4 and ASC4, despite evidence from western blotting that AIC4 gives higher yCD expression (FIG. 13). This could indicate that low levels of enzyme are sufficient for production of toxic amounts of 5-FU, or it may simply reflect the longer duration of the CPE assay.

Inspection of the cultures revealed that 5-FC increased the toxicity of the yCD viruses as soon as two days after infection. To test whether it was better to give the drug after completion of the first cycles of viral replication, we compared addition of 5-FC either directly after infection (FIG. 15f, “E”) or four days later (FIG. 15f, “L”). Late administered 5-FC was not toxic, except for a small effect with the AIC4 virus, which expresses the highest amount of yCD.

Finally, we tested the toxicity of the viruses in normal cells (HLFs, FIG. 15g). The Tcf viruses were ˜1000-fold less toxic than wild-type adenovirus type 5. AIC4 and ASC4 started to show some CPE at an moi of 10. This correlates with the expression of DBP and fibre seen in FIG. 13c. 5-FC had a marginal effect with AIC4, perhaps reflecting yCD expression below the limit of detection by western blotting (FIG. 13c).

In conclusion, 5-FC treatment of colon cancer cells infected with an oncolytic virus expressing yCD from the major late promoter increases the cytopathic effect of the virus by about 10-fold but has only a minor effect in normal cells.. The magnitude of the improvement appears small because Tcf viruses are already highly active in these cells. In SW480, for example, the parental virus can kill the cells at an moi of 0.01. The largest gain in activity was in HT29, which are relatively resistant to Tcf viruses because of low Tcf activity. The gain in activity can be unequivocally attributed to the action of yCD on 5-FC, because both are required to see the effect. It is a complex phenomenon resulting from the combination of multiple competing factors. Minimally, these include the efficiency of conversion of 5-FC to 5-FU, the sensitivity of viral and cellular replication to 5-FU, the toxicity of 5-FU and perhaps bystander effects. The increased activity in CPE assays was only seen after prolonged exposure to 5-FC, suggesting that in this experimental setting either the conversion of 5-FC to 5-FU is slow or the gain from toxicity of 5-FU outweighs its effect on viral replication.

Measurement of Viral Burst Size in the Presence of 5-FC

The complete cytopathic effect seen at low multiplicity of infection (<1 pfu/cell) suggests that 5-FC does not prevent viral spread. To directly test whether 5-FC interferes with infectious virus production, we performed burst assays on colon cancer cell lines (FIG. 5). The cell pellet and culture supernatant were tested separately to detect any effect on virus release. Viral burst size was higher in SW480 with all three viruses, as expected. In the absence of 5-FC, the yCD viruses were at least as active as A4, despite their higher particle to pfu ratio. Less virus was detected in the pellet fraction in Hct116 and HT29 after 5-FC treatment. This was compensated to some extent by virus in the supernatant, resulting in comparable total yields of virus before and after 5-FC treatment, but the difference was small and does not provide convincing evidence for an effect on virus release. We conclude that early treatment with 5-FC is fully compatible with productive infection by our yCD viruses.

Materials and Methods used in Example 7:

Adenovirus Mutagenesis

The fibre region (nucleotides nt 30470 to 33598) of adenovirus 5 (ATCC VR5) was cut with KpnI/XbaI and cloned into pUC19 to give pCF159. A SpeI site was inserted after the polyA site of the fibre by inverse PCR with primers

| | |
|---|---|
| AGTTTCTTTATTCTTGGGCAATGT | (oCF67) |
| and | |
| AGTCGTTTGTGTTATGTTTCAAC. | (oCF68) to give pCF277 |

yCD was cloned from S. cerevisiae genomic DNA by PCR with primers

TCGCTAGCCAGGCACAATCTTCGCATTTCTTTTTTTCCAGATGGTGACAG
GGGGAATGGC (oCF31)
and
TGACTAGTTATTCACCAATATCTTCAAA (oCF32).

The product was cut with NheI and SpeI (underlined) and inserted into the XbaI site of pycDNA3 (Invitrogen, Carlsbad, USA) to give pCF232.

The EMCV internal ribosome entry site (IRES) was cloned by PCR from the pSE280-IRES plasmid (gift of O. Zillian, ISREC). This plasmid contains the EMCV IRES of pCITE-1 (Novagen, Madison, USA) cut with EcoRI and BalI and cloned into the EcoRI/SmaI sites of pSE280 (Invitrogen, Carlsbad, USA). The IRES was amplified with primers ATGCTAGCGAATTCCGCCCCTCTC (oCF69) and ATACTAGTTATGCATATTATCATCGTGTTT (oCF70), cut with NheI and SpeI (underlined) and inserted into the SpeI engineered immediately downstream of the fibre to give pCF274. This plasmid contains the full-length wild-type fibre followed by the EMCV IRES. The BfrBI site at the end of the IRES (bold) can be used to introduce a foreign gene, whose first codon is the ATG of the BfrBI site. The polyA site of fibre is embedded at the end of the coding sequence and was mutated by silent mutations (GAA TAA A to GAG TAG A, where the coding sequence remains Glu-Stop). To do so, the 5′-end of the fibre gene was amplified by PCR from pCF274 using primers GGAATTCGCTAGTTTCTCTACTCTTGGGCAATGTA (oCF77, contains the mutant polyA signal, underlined) and GGTGGTGGAGATGCTAAACTCACTTTGGTC (oKH9) and re-introduced into pCF274 using EcoRI and BstXI. The vector obtained after backcloning is pCF328. It contains the full-length wild-type fibre sequence with a mutant polyA site followed by the EMCV IRES. This viral sequence is in a pRS406 backbone, see Gagnebin J et al. Gene Ther 1999; 6: 1742-1750.

yCD was cloned by PCR with primers GTGACAGGGGGAATGGCAAG (oCF71) and TGACTAGTTTATTCACCAATATCTTCAAA (oCF76), cut with SpeI and inserted into the BfrBI/SpeI sites of pCF278, a K7-fibre but otherwise identical derivative of pCF274, to give pCF308. An extra A (bold) was added at the end of yCD (last two codons underlined) to create a polyA signal. The junction between the IRES and yCD was corrected by PCR to give pCF317. The IRES-yCD cassette of pCF317 was backcloned with AvrII and SpeI into pCF328 to obtain pCF330, the corresponding shuttle vector.

The splice acceptor sequence was synthesised in oCF31 and used with oCF76 to amplify yCD by PCR. The product was cut with NheI and Spel and cloned into the SpeI site of pCF277 to give pCF298. The splice cassette of pCF298 was backcloned with XbaI and SpeI into pCF328 to obtain pCF317, the corresponding yeast integrating vector.

The IRES-yCD (pCF330) or splice-yCD (pCF317) sequences were introduced into the vCF11 (A4) YAC/BAC by two-step gene replacement in yeast to obtain vpCF12 and vpCF13, respectively. Plasmids were checked by automated fluorescent sequencing on a Licor 4200L sequencer in the fibre region using primers IF272 (Fibre sense: GCCATTAATGCAGGAGATG) and IR281 (E4 antisense: GGAGAAAGGACTGTGTACTC).

Viral genomic DNA was converted into virus by transfection of PacI digested YAC/BAC DNA into cR1 cells. The viruses were then plaque purified on SW480 cells, expanded on SW480, purified by CsCl banding, buffer exchanged using NAP25 columns into 1 M NaCl, 100 mM Tris-HCl pH 8.0, 10% glycerol and stored frozen at −70° C. The identity of each batch was checked by restriction digestion. Particle counts were based on the OD260 of virus in 0.1% SDS using the formula I OD260=1012 particles/ml. Pfu titres were measured on SW480.6 The clone names for AIC4 and ASC4 are vCF125 and vCF132, respectively.

Cell Lines

SW480 (ATCC CCL-228), HCT116 (CCL-247) and HT29 (HTB-38) were supplied by ATCC. Human embryonic lung fibroblasts (HLFs) were supplied by Dr M Nabholz. p53-/- HCT116 were supplied by Dr B Vogelstein, NEED TO ADD TO REF LIST see Bunz F et al. Science 1998; 282: 1497-1501. cR1 cells are C7 cells expressing myc-tagged ΔN-β-catenin (see above). Cell lines were grown in Dulbecco's Modified Eagle's Medium (DMEM) with 10% foetal calf serum (Invitrogen, Carlsbad, USA).

Western Blotting

Cells were infected with 10 plaque forming units (pfu) per cell in DMEM. Two hours after infection, the medium was replaced with complete medium plus or minus 20 μg/ml of cytosine arabinoside (Sigma, St. Louis, USA). Cells were harvested at various times in SDS-PAGE sample buffer. E1A, DBP, Fibre and yCD were detected with the M73 (Santa Cruz Biotechnology, Santa Cruz, USA), B6,27 4D2 (Research Diagnostics Inc, Flanders, USA) and 2485-4906 (Biogenesis, Poole, England) antibodies, respectively.

Cytopathic Effect Assay

Cells in six-well plates were infected with ten-fold dilutions of virus in DMEM. Two hours after infection, the medium was replaced with complete medium containing or not 100 μg/ml of 5-fluorocytosine (Sigma, St. Louis, USA). Four days after infection, new medium was added. Late addition of 5-FC was performed at that time. After five to eight days (see legend to FIG. 4), the cells were fixed with 4% formaldehyde in PBS and stained with crystal violet. For the sensitivity to 5-fluorouracil (Sigma, St. Louis, USA), the drug was added at various concentrations for four days before staining with crystal violet.

Virus Replication Assay

Cells in six-well plates were infected with 1 pfu per cell in DMEM. Two hours after infection, the medium was replaced with complete medium containing or not 100 μg/ml 5-fluorocytosine (5-FC). 48 hours later, the medium and the cells were collected and centrifuged at 3000 rpm in a table-top centrifuge. The supernatant was collected, while the pellet was resuspended in medium containing 10% glycerol and lysed by three cycles of freeze-thawing. Cell extracts were obtained after centrifugation of the cellular debris. Both supernatant and cell extract were tested for virus production by counting plaques formed on SW480 cells after 10 days under 0.9% Bacto agar in DMEM 10% FCS. Two independent infections were tested in triplicate for the cell extracts. One infection was tested in duplicate for the supernatant. Each bar in the figure represents the mean +/− SD.

Northern Blotting and RT-PCR

HT29 cells were infected with 10 pfu per cell in DMEM. RNA was extracted with the Qiagen Rneasy midi kit following the manufacturer's instructions (Qiagen, Hilden, D). 10 μg total RNA per lane was resolved on a 1.2% agarose/1× MOPS/6.3% formaldehyde gel. RNA was transferred by capillarity with 20×SSC on positively charged membrane (Appligene, Strasbourg, France) and UV cross-linked to the membrane in a Stratalinker (Stratagene, La Jolla, USA). Northern blots were hybridised with random-primed 32P-labeled probes corresponding to full-length cytosine deaminase (482 bp, PCR with oCF71 and oCF76) or a 468 bp fragment of fibre (NheI to HindIII). The membranes were prehybridised in Church Buffer (0.5M NaPO4, 1 mM EDTA, 7% SDS, 1% BSA) for 2 hours at 65° C. and hybridised in the same conditions overnight. Blots were washed in 2×SSC, 0.1% SDS at 65° C., and then in 1×SSC, 0.1% SDS at 65° C.

RT was performed with oligo-dT12-18 (Amersham Biosciences, Little Chalfont, UK) and Superscript II reverse transcriptase (Invitrogen, Carlsbad, USA) according to the manufacturer's instructions. yCD was amplified with Pfu turbo (Stratagene, La Jolla, USA) using primers oCF76 and AGGATCCACTCTCTTCCGCATCGCTGTC (TPLupper). Bands were purified from a TAE agarose gel and 3′ A-Overhangs were added with Taq DNA Polymerase (Sigma, St. Louis, USA). The PCR product was cloned by TOPO TA cloning into pCR2-1-TOPO following the manufacturer's instructions (Invitrogen, Carlsbad, USA) and sequenced using primers AGGGTTTTCCCAGTCACGACGTT (M13fwd) and AGCGGATAACAATTTCACACAGGA (M13rev).

The following references for procedures are incorporated herein by reference:

Bouton, A. H., and Smith, M. M. (1986). Fine-structure analysis of the DNA sequence requirements for autonomous replication of Saccharomyces cerevisiae plasmids. Mol Cell Biol 6, 2354-63.

Ketner, G., Spencer, F., Tugendreich, S., Connelly, C., and Hieter, P. (1994). Efficient manipulation of the human adenovirus genome as an infectious yeast artificial chromosome clone. Proc Natl Acad Sci U S A 91, 6186-90.

Larionov, V., Kouprina, N., Graves, J., Chen, X. N., Korenberg, J. R., and Resnick, M. A. (1996). Specific cloning of human DNA as yeast artificial chromosomes by transformation-associated recombination. Proc Natl Acad Sci U S A 93, 491-6.

Promoter replacement sequences inserts for preparing Ad-Tcf viruses single Tcf site:

ATCAAAGGG

2 Tcf sites:

ATCAAAGGGATCCAGATCAAAGG-

3 Tcf sites:

ATCAAGGGTTGGAGATCAAAGGGATCCAGATCAAAGGGATTAA
GAT CAAAGG-

4 Tcf sites:

-ATCAAAGGGTTGGAGATCAAAGGGATCCAGATCAAAGGGATTA
AGATCAAAGG-

References
1. Alevizopoulos, K., B. Catarin, J. Vlach, and B. Amati. 1998. A novel function of adenovirus E1A is required to overcome growth arrest by the CDK2 inhibitor p27(Kip1). EMBO J. 17:5987-97.
2. Alevizopoulos, K., B. Sanchez, and B. Amati. 2000. Conserved region 2 of adenovirus E1A has a function distinct from pRb binding required to prevent cell cycle arrest by p16INK4a or p27Kip1. Oncogene. 19:2067-74.
3. Amalfitano, A., and J. S. Chamberlain. 1997. Isolation and characterization of packaging cell lines that coexpress the adenovirus E1, DNA polymerase, and preterminal proteins: implications for gene therapy. Gene Ther. 4:258-63.
4. Bartek, J., Bartkova, J., and Lukas, J. (1997). The retinoblastoma protein pathway in cell cycle control and cancer. Exp Cell Res 237, 1-6.
5. Bauer, A., S. Chauvet, O. Huber, F. Usseglio, U. Rothbacher, D. Aragnol, R. Kemler, and J. Pradel. 2000. Pontin52 and Reptin52 function as antagonistic regulators of beta-catenin signalling activity. EMBO Journal. 19:6121-6130.
6. Bischoff, J. R., D. H. Kirn, A. Williams, C. Heise, S. Horn, M. Muna, L. Ng, J. A. Nye, A. Sampson-Johannes, A. Fattaey, and F. McCormick. 1996. An adenovirus mutant that replicates selectively in p53-deficient human tumor cells. Science. 274:373-6.
7. Blanco, J. C., S. Minucci, J. Lu, X. J. Yang, K. K. Walker, H. Chen, R. M. Evans, Y. Nakatani, and K. Ozato. 1998. The histone acetylase PCAF is a nuclear receptor coactivator. Genes Dev. 12:1638-51.
8. Bonneaud, N., K. O. Ozier, G. Y. Li, M. Labouesse, S. L. Minvielle, and F. Lacroute. 1991. A family of low and high copy replicative, integrative and single-stranded S. cerevisiae/E. coli shuttle vectors. Yeast. 7:609-15.
9. Brannon, M., J. D. Brown, R. Bates, D. Kimelman, and R. T. Moon. 1999. XCtBP is a XTcf-3 co-repressor with roles throughout Xenopus development. Development. 126:3159-70.
10. Brunori, M., M. Malerba, H. Kashiwazaki, and R. Iggo. 2001. Replicating adenoviruses that target tumors with constitutive activation of the wnt signaling athway. J Virol. 75:2857-65.
11. Cajot, J. F., I. Sordat, T. Silvestre, and B. Sordat. 1997. Differential display cloning identifies motility-related protein (MRP1/CD9) as highly expressed in primary compared to metastatic human colon carcinoma cells. Cancer Res. 57:2593-7.
12. Challberg, M. D., and T. J. Kelly. 1989. Animal virus DNA replication. Annu Rev Biochem. 58:671-717.
13. Chen, X., L. J. Ko, L. Jayaraman, and C. Prives. 1996. p53 levels, functional domains, and DNA damage determine the extent of the apoptotic response of tumor cells. Genes Dev. 10:2438-51.
14. Chen, Y., D. C. Yu, C. D., and D. R. Henderson. 2000. Pre-existent adenovirus antibody inhibits systemic toxicity and antitumor activity of CN706 in the nude mouse LNCAP xenograft model: Implications and proposals for human therapy. Hum Gene Ther. 11: 1553-1567.
15. Cottu, P. H., F. Muzeau, A. Estreicher, J. F. Flejou, R. Iggo, G. Thomas, and R. Hamelin. 1996. Inverse correlation between RER+status and p53 mutation in colorectal cancer cell lines. Oncogene. 13:2727-30.
16. Crawford, H. C., B. M. Fingleton, L. A. Rudolph-Owen, K. J. Goss, B. Rubinfeld, P. Polakis, and L. M. Matrisian. 1999. The metalloproteinase matrilysin is a target of beta-catenin transactivation in intestinal tumors. Oncogene. 18:2883-91.
17. Chartier, C., Degryse, E., Gantzer, M., Dieterle, A., Pavirani, A., and Mehtali, M. (1996). Efficient generation of recombinant adenovirus vectors by homologous recombination in Escherichia coli. J Virol 70, 4805-10.
18. Clayman, G. L., el-Naggar, A. K., Lippman, S. M., Henderson, Y. C., Frederick, M., Merritt, J. A., Zumstein, L. A., Timmons, T. M., Liu, T. J., Ginsberg, L., Roth, J. A., Hong, W. K., Bruso, P., and Goepfert, H. (1998). Adenovirus-mediated p53 gene transfer in patients with advanced recurrent head and neck squamous cell carcinoma. J Clin Oncol 16, 2221-32.

19. Croyle, M. A., Stone, M., Amidon, G. L., and Roessler, B. J. (1998). In vitro and in vivo assessment of adenovirus 41 as a vector for gene delivery to the intestine. Gene Ther 5, 645-654.
20. Crystal, R. G., Hirschowitz, E., Lieberman, M., Daly, J., Kazam, E., Henschke, C., Yankelevitz, D., Kemeny, N., Silverstein, R., Ohwada, A., Russi, T., Mastrangeli, A., Sanders, A., Cooke, J., and Harvey, B. G. (1997). Phase I study of direct administration of a replication deficient adenovirus vector containing the E. coli cytosine deaminase gene to metastatic colon carcinoma of the liver in association with the oral administration of the pro-drug 5-fluorocytosine. Hum Gene Ther 8, 985-1001.
21. Cunningham, D., Pyrhönen, S., James, R. D., Punt, C. J. A., Hickish, T. S., Heikkila, R., Johannesen, T., Starkhammar, H., Topham, C. A., Ong, E., Herait, P., and Jacques, C. (1998). A phase III multicenter randomized study of CPT-11 versus supportive care alone in patients with 5FU-resistant metastatic colorectal cancer. Proc Am Soc Clin Oncol 17, abstract 1.
22. Dahmane, N., Lee, J., Robins, P., Heller, P., and Ruiz i Altaba, A. (1997). Activation of the transcription factor Glil and the Sonic hedgehog signalling pathway in skin tumors. Nature 389, 876-81.
23. Ellisen, L. W., Bird, J., West, D. C., Soreng, A. L., Reynolds, T. C., Smith, S. D., and Sklar, J. (1991). TAN-1, the human homolog of the Drosophila notch gene, is broken by chromosomal translocations in T lymphoblastic neoplasms. Cell 66, 649-61.
24. Estreicher, A., and Iggo, R. (1996). Retrovirus-mediated p53 gene therapy. Nature Med 2, 1163.
25. Evan, G. I., G. K. Lewis, G. Ramsay, and J. M. Bishop. 1985. Isolation of monoclonal antibodies specific for human c-myc proto-oncogene product. Mol Cell Biol. 5:3610-6.
26. Fajas, L., C. Paul, O. Zugasti, L. Le Cam, J. Polanowska, E. Fabbrizio, R. Medema, M. L. Vignais, and C. Sardet. 2000. pRB binds to and modulates the transrepressing activity of the E1A-regulated transcription factor pl2OE4F. Proc Natl Acad Sci U S A. 97:7738-43.
27. Fisher, A. L., and Caudy, M. (1998). Groucho proteins: transcriptional corepressors for specific subsets of DNA-binding transcription factors in vertebrates and invertebrates. Genes Dev 12, 1931-40.
28. Flaman, J. M., Frebourg, T., Moreau, V., Charbonnier, F., Martin, C., Ishioka, C., Friend, S. H., and Iggo, R. (1994). A rapid PCR fidelity assay. Nucleic Acids Res 22, 3259-60.
29. Gagnebin, J., M. Brunori, M. Otter, L. Juillerat-Jeaneret, P. Monnier, and R. Iggo. 1999. A photosensitising adenovirus for photodynamic therapy. Gene Ther. 6:1742-1750.
30. Gagnebin, J., Kovar, H., Kajava, A. V., Estreicher, A., Jug, G., Monnier, P., and Iggo, R. (1998). Use of transcription reporters with novel p53 binding sites to target tumor cells expressing endogenous or virally transduced p53 mutants with altered sequence-specificity. Oncogene 16, 685-90.
31. Hallenbeck, P. L., Y. N. Chang, C. Hay, D. Golightly, D. Stewart, J. Lin, S. Phipps, and Y. L. Chiang. 1999. A novel tumor-specific replication-restricted adenoviral vector for gene therapy of hepatocellular carcinoma. Human Gene Therapy. 10:1721-33.
32. He, T. C., A. B. Sparks, C. Rago, H. Hermeking, L. Zawel, L. T. da Costa, P. J. Morin, B. Vogelstein, and K. W. Kinzler. 1998. Identification of c-MYC as a target of the APC pathway. Science. 281:1509-12.
33. Hearing, P., and T. Shenk. 1986. The adenovirus type 5 E1A enhancer contains two functionally distinct domains: one is specific for E1A and the other modulates all early units in cis. Cell. 45:229-36.
34. Hecht, A., K. Vleminckx, M. P. Stemmler, F. van Roy, and R. Kemler. 2000. The p300/CBP acetyltransferases function as transcriptional coactivators of beta-catenin in vertebrates. EMBO J. 19:1839-50.
35. Heise, C., T. Hermiston, L. Johnson, G. Brooks, A. Sampson-Johannes, A. Williams, L. Hawkins, and D. Kirn. 2000. An adenovirus E1A mutant that demonstrates potent and selective systemic anti-tumoral efficacy. Nature Med. 6:1134-1139.
36. Heise, C., A. Sampson-Johannes, A. Williams, F. McCormick, D. D. Von Hoff, and D. H. Kirn. 1997. ONYX-015, an E1B gene-attenuated adenovirus, causes tumor-specific cytolysis and antitumoral efficacy that can be augmented by standard chemotherapeutic agents. Nature Med. 3:639-645.
37. Hernandez-Alcoceba, R., M. Pihalja, M. S. Wicha, and M. F. Clarke. 2000. A novel, conditionally replicative adenovirus for the treatment of breast cancer that allows controlled replication of Ela-deleted adenoviral vectors. Human Gene Therapy. 11:2009-2024.
38. Kemeny, N., Daly, J., Reichman, B., Geller, N., Botet, J., and Oderman, P. (1987). Intrahepatic or systemic infusion of fluorodeoxyuridine in patients with liver metastases from colorectal carcinoma. A randomized trial. Ann Intern Med 107, 459-65.
39. Kemeny, N., Seiter, K., Niedzwiecki, D., Chapman, D., Sigurdson, E., Cohen, A., Botet, J., Oderman, P., and Murray, P. (1992). A randomized trial of intrahepatic infusion of fluorodeoxyuridine with dexamethasone versus fluorodeoxyuridine alone in the treatment of metastatic colorectal cancer. Cancer 69, 327-34.
40. Ketner, G., Spencer, F., Tugendreich, S., Connelly, C., and Hieter, P. (1994). Efficient manipulation of the human adenovirus genome as an infectious yeast artificial chromosome clone. Proc Natl Acad Sci U S A 91, 6186-90.
41. Korinek, V., Barker, N., Morin, P. J., van Wichen, D., de Weger, R., Kinzler, K. W., Vogelstein, B., and Clevers, H. (1997). Constitutive transcriptional activation by a beta-catenin-Tcf complex in APC-/- colon carcinoma. Science 275, 1784-7.
42. Khuri, F., J. Nemunaitis, I. Ganly, J. Arseneau, I. Tannock, L. Romel, M. Gore, J. Ironside, R. MacDougall, C. Heise, B. Randlev, G. Gillenwater, P. Bruso, S. Kaye, H. W., and D. Kirn. 2000. A controlled trial of intratumoral ONYX-015, a selectively-replicating adenovirus, in combination with cisplatin and 5-fluorouracil in patients with recurrent head and neck cancer. Nature Med. 6:879 - 885.
43. Korinek, V., N. Barker, P. J. Morin, D. van Wichen, R. de Weger, K. W. Kinzier, B. Vogelstein, and H. Clevers. 1997. Constitutive transcriptional activation by a beta-catenin-Tcf complex in APC-/- colon carcinoma. Science. 275:1784-7.
44. Kraus, V. B., J. A. Inostroza, K. Yeung, D. Reinberg, and J. R. Nevins. 1994. Interaction of the Dr1 inhibitory factor with the TATA binding protein is disrupted by adenovirus E1A. Proc Natl Acad Sci U S A. 91:6279-82.
45. Jarriault, S., Brou, C., Logeat, F., Schroeter, E. H., Kopan, R., and Israel, A. (1995). Signalling downstream of activated mammalian Notch. Nature 377, 355-8.
46. Labianca, R., Pessi, M. A., and Zamparelli, G. (1997). Treatment of colorectal cancer. Current guidelines and future prospects for drug therapy. Drugs 53, 593-607.
47. Leyvraz, S., Spataro, V., Bauer, J., Pampallona, S., Salmon, R., Dorval, T., Meuli, R., Gillet, M., Lejeune, F., and Zografos, L. (1997). Treatment of ocular melanoma metastatic to the liver by hepatic arterial chemotherapy. J Clin Oncol 15, 2589-95.
48. Lee, J. S., K. M. Galvin, R. H. See, R. Eckner, D. Livingston, E. Moran, and Y. Shi. 1995. Relief of YY1 transcriptional repression by adenovirus E1A is mediated by E1A-associated protein p300. Genes Dev. 9:1188-98.
49. Leza, M. A., and P. Hearing. 1988. Cellular transcription factor binds to adenovirus early region promoters and to a cyclic AMP response element. J Virol. 62:3003-13.
50. Lipinski, K. S., H. Esche, and D. Brockmann. 1998. Amino acids 1-29 of the adenovirus serotypes 12 and 2 E1A proteins interact with rap30 (TF(II)F) and TBP in vitro. Virus Res. 54:99-106.
51. Lewis, B. A., G. Tullis, E. Seto, N. Horikoshi, R. Weinmann, and T. Shenk. 1995. Adenovirus E1A proteins interact with the cellular YY1 transcription factor. J Virol. 69:1628-36.
52. Marton, M. J., S. B. Baim, D. A. Ornelles, and T. Shenk. 1990. The adenovirus E4 17-kilodalton protein complexes with the cellular transcription factor E2F, altering its DNA-binding properties and stimulating E1A-independent accumulation of E2 mRNA. J Virol. 64:2345-59.
53. Morin, P. J., Sparks, A. B., Korinek, V., Barker, N., Clevers, H., Vogelstein, B., and Kinzler, K. W. (1997). Activation of beta-catenin-Tcf signaling in colon cancer by mutations in beta-catenin or APC. Science 275, 1787-90.
54. Ogawa, N., Fujiwara, T., Kagawa, S., Nishizaki, M., Morimoto, Y., Tanida, T., Hizuta, A., Yasuda, T., Roth, J. A., and Tanaka, N. (1997). Novel combination therapy for human colon cancer with adenovirus- mediated wild-type p53 gene transfer and DNA-damaging chemotherapeutic agent. Int J Cancer 73, 367-70.
55. Parr, M. J., Manome, Y., Tanaka, T., Wen, P., Kufe, D. W., Kaelin, W. G., and Fine, H. A. (1997). Tumor-Selective Transgene Expression In Vivo Mediated By an E2f-Responsive Adenoviral Vector. Nature Medicine 3, 1145-1149.
56. Patt, Y. Z., and Mavligit, G. M. (1991). Arterial chemotherapy in the management of colorectal cancer: an overview. Semin Oncol 18, 478-90.
57. Polakis, P. 2000. Wnt signaling and cancer. Genes & Dev. 14:1837-1851.
58. Qian, C., Bilbao, R., Bruna, O., and Prieto, J. (1995). Induction of sensitivity to ganciclovir in human hepatocellular carcinoma cells by adenovirus-mediated gene transfer of herpes simplex virus thymidine kinase. Hepatology 22, 118-23.
59. Reich, N. C., P. Sarnow, E. Duprey, and A. J. Levine. 1983. Monoclonal antibodies which recognize native and denatured forms of the adenovirus DNA-binding protein. Virology. 128:480-4.
60. Reid, J. L., A. J. Bannister, P. Zegerman, M. A. Martinez-Balbas, and T. Kouzarides. 1998. E1A directly binds and regulates the P/CAF acetyltransferase. EMBO J. 17:4469-77.
61. Rodriguez, R., E. R. Schuur, H. Y. Lim, G. A. Henderson, J. W. Simons, and D. R. Henderson. 1997. Prostate attenuated replication competent adenovirus (ARCA) CN706: a selective cytotoxic for prostate-specific antigen-positive prostate cancer cells. Cancer Research. 57:2559-63.
62. Roth, J. A., and Cristiano, R. J. (1997). Gene therapy for cancer: what have we done and where are we going? J Natl Cancer Inst 89, 21-39.
63. Roth, J. A., Nguyen, D., Lawrence, D. D., Kemp, B. L., Carrasco, C. H., Ferson, D. Z., Hong, W. K., Komaki, R., Lee, J. J., Nesbitt, J. C., Pisters, K. M., Putnam, J. B., Schea, R., Shin, D. M., Walsh, G. L., Dolormente, M. M., Han, C. I., Martin, F. D., Yen, N., Xu, K., Stephens, L. C., McDonnell, T. J., Mukhopadhyay, T., and Cai, D. (1996). Retrovirus-mediated wild-type p53 gene transfer to tumors of patients with lung cancer. Nature Med 2, 985-991.
64. Rubinfeld, B., Robbins, P., El-Gamil, M., Albert, I., Porfiri, E., and Polakis, P. (1997). Stabilization of beta-catenin by genetic defects in melanoma cell lines. Science 275, 1790-2.
65. Roose, J., and H. Clevers. 1999. TCF transcription factors: molecular switches in carcinogenesis. BBA. 1424:M23-37.
66. Roose, J., G. Huls, M. van Beest, P. Moerer, K. van der Horn, R. Goldschmeding, T. Logtenberg, and H. Clevers. 1999. Synergy between tumor suppressor APC and the beta-catenin-Tcf4 target Tcfl. Science. 285:1923-6.
67. Sandig, V., Brand, K., Herwig, S., Lukas, J., Bartek, J., and Strauss, M. (1997). Adenovirally transferred pl6INK4/CDKN2 and p53 genes cooperate to induce apoptotic tumor cell death. Nat Med 3, 313-9.
68. Shenk, T. (1996). Adenoviridae: the viruses and their replication. In Fields Virology, D. M. K. B.N. Fields, P.M. Howley et al., ed. (Philadelphia: Lippincott-Raven Publishers), pp. 2111-2148.
69. Stevenson, S. C., Rollence, M., Marshall-Neff, J., and McClelland, A. (1997). Selective targeting of human cells by a chimeric adenovirus vector containing a modified fiber protein. J Virol 71, 4782-90.
70., Stone, D. M., Hynes, M., Armanini, M., Swanson, T. A., Gu, Q., Johnson, R. L., Scott, M. P., Pennica, D., Goddard, A., Phillips, H., Noll, M., Hooper, J. E., de Sauvage, F., and Rosenthal, A. (1996). The tumor-suppressor gene patched encodes a candidate receptor for Sonic hedgehog. Nature 384, 129-34.
71. Stupp, R., Focan, C., Sessa, C., Nowacki, M., Vindevoghel, A., Canon, J. L., Zaluski, J., Oulid-Aissa, D., Boussard, B., and Pestalozzi, B. (1998). Prophylactic use of antibiotics during treatment with CPT-1 1 for metastatic colorectal cancer. A randomized multicenter trial. Ann Oncol in press.
72. Sarnow, P., C. A. Sullivan, and A. J. Levine. 1982. A monoclonal antibody detecting the adenovirus type 5-Elb-58Kd tumor antigen: characterization of the Elb-58 Kd tumor antigen in adenovirus-infected and -transformed cells. Virology. 120:510-7.
73. Sun, Y., F. T. Kolligs, M. O. Hottiger, R. Mosavin, E. R. Fearon, and G. J. Nabel. 2000. Regulation of beta -catenin transformation by the p300 transcriptional coactivator. Proc Natl Acad Sci U S A. 97:12613-8.
74. Takemaru, K. I., and R. T. Moon. 2000. The transcriptional coactivator CBP interacts with beta-catenin to activate gene expression. J Cell Biol. 149:249-54.
75. Teichler, Z. D. 2000. US gene therapy in crisis. Trends Genet. 16:272-5.
76. Tetsu, O., and F. McCormick. 1999. Beta-catenin regulates expression of cyclin D1 in colon carcinoma cells. Nature. 398:422-6.
77. van de Wetering, M., R. Cavallo, D. Dooijes, M. van Beest, J. van Es, J. Loureiro, A. Ypma, D. Hursh, T. Jones, A. Bejsovec, M. Peifer, M. Mortin, and H. Clevers. 1997. Armadillo coactivates transcription driven by the product of the Drosophila segment polarity gene dTCF. Cell. 88:789-99.
78. Van der Eb, M. M., Cramer, S. J., Vergouwe, Y., Schagen, F. H., van Krieken, J. H., van der Eb, A. J., Rinkes, I. H., van de Velde, C. J., and Hoeben, R. C. (1998). Severe hepatic dysfunction after adenovirus-mediated transfer of the herpes simplex virus thymidine kinase gene and ganciclovir administration. Gene Ther 5, 451-8.
79. Wood, M. A., S. B. McMahon, and M. D. Cole. 2000. An ATPase/helicase complex is an essential cofactor for oncogenic transformation by c-Myc. Mol Cell. 5:321-30.
80. Xie, J., Murone, M., Luoh, S. M., Ryan, A., Gu, Q., Zhang, C., Bonifas, J. M., Lam, C. W., Hynes, M., Goddard, A., Rosenthal, A., Epstein, E. H., Jr., and de Sauvage, F. J. (1998). Activating Smoothened mutations in sporadic basal-cell carcinoma. Nature 391, 90
81. Yu, D. C., G. T. Sakamoto, and D. R. Henderson. 1999. Identification of the transcriptional regulatory sequences of human kallikrein 2 and their use in the construction of calydon virus 764, an attenuated replication competent adenovirus for prostate cancer therapy. Cancer Res. 59:1498-504.
82. Meta-Analysis Group in Cancer, (1996). Reappraisal of hepatic arterial infusion in the treatment of nonresectable liver metastases from colorectal cancer. J Natl Cancer Inst 88, 252-8.

83. Gagnebin J et al. A photosensitising adenovirus for photodynamic therapy. Gene Ther 1999; 6: 1742-1750.
84. Sauthoff H et al. Late expression of p53 from a replicating adenovirus improves tumor cell killing and is more tumor cell specific than expression of the adenoviral death protein. Hum Gene Ther 2002; 13: 1859-1871.
85 Wong ET, Ngoi SM, Lee CG. Improved co-expression of multiple genes in vectors containing internal ribosome entry sites (IRESes) from human genes. Gene Ther 2002; 9: 337-344.

1. A viral DNA construct encoding for an adenovirus capable of replication in a human or animal tumor cell comprising one or more selected transcription factor binding sites operatively positioned together with the E1A open reading frame such as to promote expression of E1A proteins in the presence of said selected transcription factor, wherein the level or activity of the transcription factor is increased in a human or animal tumor cell relative to that of a normal human or animal cell of the same type; and wherein the viral DNA construct further comprises a therapeutic gene.

2. A viral construct as claimed in claim 1 wherein the therapeutic gene is a suicide gene positioned between the fibre gene and the E4 region in the major late transcription unit of the viral construct.

3. A viral construct as claimed in claim 1 wherein the construct encodes a full complement of adenoviral proteins.

4. A viral construct as claimed in claim 1 wherein the wild type packaging signal is deleted from its wild type site adjacent the left hand inverted terminal repeat (ITR) and inserted elsewhere in the construct, in either forward or backward orientation.

5. A viral construct as claimed in claim 2 wherein the suicide gene is selected from HSV thymidine kinase, nitroreductase and cytosine deaminase.

6. A viral construct as claimed in claim 1 wherein the therapeutic gene is expressed late in a replication-dependent manner using an IRES or by differential splicing.

7. A viral construct according to claim 1 wherein the selected transcription factor binding site is a Tcf-4 transcription factor binding site.

8. A viral construct as claimed in claim 1 wherein the E4 promoter contains part of the E1A enhancer of the packaging signal flanked by Tcf and E4F sites.

9. A virus comprising or encoded by the DNA construct as claimed in claim 1.

10. A method for treating a patient in need of therapy for a neoplasm wherein a viral DNA construct as claimed claim 1 is caused to infect tissues of the patient, including or restricted to those of the neoplasm, and allowed to replicate such that neoplasm cells are caused to be killed.

11. A method as claimed in claim 10 characterised in that the patient is in need of therapy for a colon cell derived tumor.

12. A viral DNA construct encoding for an adenovirus capable of replication in a human or animal tumor cell comprising one or more selected tumor specific transcription factor binding sites replacing one of more wild type transcription factor binding sites in the viral promoter sequences such as to control expression of viral genes, wherein the level or activity of the tumor specific transcription factor is increased in a human or animal tumor cell relative to that of a normal human or animal cell of the same type; and a therapeutic gene; and wherein the viral construct encodes a full complement of wild type genes.

13. A viral construct as claimed in claim 12 wherein the selected transcription factor binding sites are operatively positioned together with the E1A open reading frame such as to promote expression of E1A proteins in the presence of said selected transcription factor.

14. A viral construct as claimed in claim 12 wherein the wild type packaging signal is deleted from its wild type site adjacent the left hand inverted terminal repeat (ITR) and inserted elsewhere in the construct, in either forward or backward orientation.

15. A viral construct as claimed in claim 12 wherein the selected transcription factor binding sites are single or multiple Tcf-4 binding sites.

16. A viral construct as claimed in claim 12 wherein the therapeutic gene is expressed in a replication-dependent manner from the major late transcription unit.

17. A virus comprising or encoded by the DNA construct as claimed in claim 12.

18. A method for treating a patient in need of therapy for a neoplasm wherein a viral DNA construct as claimed in claim 12 is caused to infect tissues of the patient, including or restricted to those of the neoplasm, and allowed to replicate such that neoplasm cells are caused to be killed.

19. A method as claimed in claim 18 characterised in that the patient is in need of therapy for a colon cell derived tumor.

20. A viral DNA construct encoding for an adenovirus capable of replication in a human or animal tumor cell comprising one or more selected transcription factor binding sites operatively positioned together with one or more of the E1B, E2 and E3 open reading frames such as to promote expression of one or more E1B, E2 and E3 proteins in the presence of said selected transcription factor, wherein the level or activity of the transcription factor is increased in a human or animal tumor cell relative to that of a normal human or animal cell of the same type; and a therapeutic gene.

21. A viral construct according to claim 20 wherein the selected transcription factor binding sites are selected from Tcf-4, RBPJK, Gli-1, HIF1 alpha and telomerase promoter binding sites.

22. A viral construct according to claim 20 wherein the therapeutic gene is a suicide gene expressed in a replication-dependent manner.

23. A viral construct as claimed in claim 20 wherein the therapeutic gene is positioned between the fibre gene and E4 in the major late transcription unit.

24. A viral construct as claimed in claim 20 wherein one or more of the selected transcription factor binding sites are inserted into the right hand terminal repeat such as to provide sufficient symmetry to allow it to base pair to the left hand ITR during replication.

25. A viral construct as claimed in claim 20 wherein the viral construct encodes a full complement of adenoviral proteins.

26. A virus comprising or encoded by the DNA construct as claimed in claim 20.

27. A method for treating a patient in need of therapy for a neoplasm wherein a viral DNA construct as claimed in claim 20 is caused to infect tissues of the patient, including or restricted to those of the neoplasm, and allowed to replicate such that neoplasm cells are caused to be killed.

28. A method as claimed in claim 27 characterised in that the patient is in need of therapy for a colon cell derived tumor.

29. A method of producing a viral DNA construct encoding for an adenovirus capable of selective replication in a human or animal tumor cell comprising removal of regions comprisng one or more wild type transcription factor binding sites from one or more viral promoters and replacement of said regions with one or more tumor specific transcription factor binding sites, wherein the replacement with tumor specific transcription factor provides spare packaging capacity in the viral construct; inserting a therapeutic gene; and retaining a full complement of wild type viral genes in the construct.

30. A method as claimed in claim 29 wherein the therapeutic gene is a suicide gene.

31. A method as claimed in claim 30 wherein the suicide gene is inserted between the fibre gene and the E4 region.
